(19) United States
(12) Patent Application Publication
(10) Pub. No.: US 2016/0338971 A1
(43) Pub. Date: Nov. 24, 2016

(54) NANO CO-ENCAPSULATION FOR THE PREVENTION AND TREATMENT OF VARIOUS DISORDERS

(71) Applicants: Shaker A. Mousa, Wynantskill, NY (US); Soad K. Jaouni, Jeddah (SA)

(72) Inventors: Shaker A. Mousa, Wynantskill, NY (US); Soad K. Jaouni, Jeddah (SA)

(21) Appl. No.: 15/011,976

(22) Filed: Feb. 1, 2016

Related U.S. Application Data
(60) Provisional application No. 62/109,867, filed on Jan. 30, 2015.

(51) Int. Cl. A61K 9/51; A61K 31/095; A61K 31/366; A61K 31/05; A61K 36/889

(52) U.S. Cl. CPC A61K 9/5161; A61K 9/5153; A61K 31/05; A61K 9/5123; A61K 36/889; A61K 31/366; A61K 31/095

(57) ABSTRACT

A nano-composition that includes nanoparticles, a method of forming the nano-composition, and a method of using the composition. The nanoparticles include a nano-shell and one or more compounds co-encapsulated within the nano-shell. The nano-shell of each nanoparticle includes one or more chitosan polymers and one or more polymers subject to each chitosan polymer being covalently bonded to the one or more polymer. Each chitosan polymer in the nano-shell of each nanoparticle includes chitosan, tri-methylated chitosan, or a combination thereof. The one or more polymers in the nano-shell of each nanoparticle include poly (lactide-co-glycolide) (PLGA), one or more fatty acids, or combinations thereof subject to each fatty acid including oligomer epigallocatechin-3-gallate (OEGCG), hyaluronic acid, oleic acids, myristic acid, caprylic acid, or combinations thereof. Each compound co-encapsulated within the nano-shell includes ajwa extracts, pomegranate extracts, garlic extracts, one or more polyphenols, one or more thiols, or combinations thereof.

### RELATED APPLICATIONS

The present invention claims priority to U.S. Provisional No. 62/109,867, filed on Jan. 30, 2015, which is incorporated herein by reference in its entirety.

### TECHNICAL FIELD

The present invention relates to a composition and associated method for use in prevention and treatment of various disorders via passive and active targeting using naturally derived targeting moieties.

### BACKGROUND

Polyphenols (e.g., flavone, isoflavone, flavonoid) derived from Ajwa dates, Green tea, Pomegranate, Raspberry, and other natural sources are potent antioxidants and chemo-preventive of various vascular, cardiovascular, oncological disorders, and infectious diseases but have low bioavailability, unstable, rapid clearance, and a short half-life. Similarly, thiols (sulforaphan, ajoene, allicin, and others) containing compounds derived from different natural products are effective antioxidants and chemo-preventive of various disorders and act by different mechanisms from that of polyphenols but are unstable and have poor bioavailability.

### BRIEF SUMMARY

The present invention provides a nano-composition that comprises nanoparticles. The nanoparticles include a nano-shell and one or more compounds co-encapsulated within the nano-shell. The nano-shell of each nanoparticle comprises one or more chitosan polymers and one or more polymers subject to each chitosan polymer being covalently bonded to the one or more polymer. Each chitosan polymer in the nano-shell of each nanoparticle is independently selected from the group consisting of chitosan, tri-methylated chitosan, and a combination thereof. The one or more polymers in the nano-shell of each nanoparticle comprise poly(lactide-co-glycolide) (PLGA), one or more fatty acids, or combinations thereof subject to each fatty acid being independently selected from the group consisting of oligomer epigallocatechin-3-gallate (OEGCG), hyaluronic acid, oleic acids, myristic acid, caprylic acid, and combinations thereof. Each compound co-encapsulated within the nano-shell is independently selected from the group consisting of ajwa extracts, pomegranate extracts, garlic extracts, one or more polyphenols, one or more thiols, and combinations thereof.

The present invention provides a method of forming the nano-composition, said method comprising: forming the nano-shell of each nanoparticle; and co-encapsulating the one or more compounds within the nano-shell of each nanoparticle.

The present invention provides a method of using the composition, said method comprising: administering the nano-composition to a human being.

### BRIEF DESCRIPTION OF THE DRAWINGS

FIG. 1 depicts SEP Fraction eluted with 30% Methanol—HPLC chromatogram and UV spectrum of date seed extract fraction through XAD-7 resin, in accordance with embodiments of the present invention.

FIG. 2 depicts SEP Fraction eluted with 45% Methanol—HPLC chromatogram and UV spectrum of date seed extract fraction through XAD-7 resin, in accordance with embodiments of the present invention.

FIG. 3 depicts SEP Fraction eluted with 60% Methanol—HPLC chromatogram and UV spectrum of date seed extract fraction through XAD-7 resin, in accordance with embodiments of the present invention.

FIG. 4 depicts SEP Fraction eluted with 70% Methanol—HPLC chromatogram and UV spectrum of date seed extract fraction through XAD-7 resin, in accordance with embodiments of the present invention.

FIG. 5 depicts SEP Fraction eluted with 100% Methanol—HPLC chromatogram and UV spectrum of date seed extract fraction through XAD-7 resin, in accordance with embodiments of the present invention.

FIG. 6 depicts a mass spectrogram of 60% ethanol extract of date seed), in accordance with embodiments of the present invention.

FIG. 7 depicts SIM (selected ion monitoring) plots of 60% ethanol extract of date seed powder, in accordance with embodiments of the present invention.

FIG. 8 depicts chemical structure and schematic illustration of OEGCG synthesized from the inter-molecular poly condensation reaction of EGCG, in accordance with embodiments of the present invention.

FIG. 9 depicts chemical structure and schematic illustration of OEGCG synthesized from the intermolecular poly condensation reaction of EGCG, in accordance with embodiments of the present invention.

FIG. 10 depicts the preparation of freeze-dried CS NPs, in accordance with embodiments of the present invention.

FIG. 11 depicts complex formation of Chitosan (CH) derivative and Hyaluronic acid derivative for encapsulation of water soluble extract or bioactive compounds thereof, in accordance with embodiments of the present invention.

FIG. 12 depicts nanoformulation of complex formation of Chitosan and Oleic acid, Myristic acid, and Caprylic acid for encapsulation of water soluble or insoluble extract and bioactive compounds thereof, in accordance with embodiments of the present invention.

FIG. 13A depicts procedures for preparation of dates flesh extract of bioactive compounds, in accordance with embodiments of the present invention.

FIG. 13B depicts procedures for preparation of dates seed extract, in accordance with embodiments of the present invention.

FIG. 13C depicts procedures for extraction and HPLC analysis for date's seed powder, in accordance with embodiments of the present invention.

FIG. 14A depicts MS-MS spectra of punicalagin: punicalin and gallagic acid, in accordance with embodiments of the present invention.

FIG. 14B depicts spectra of the molecular ion of ellagic acid, in accordance with embodiments of the present invention.

FIG. 14C depicts HPLC chromatogram of punicalagin and gallagic acid separated on C18 column, with mobile phase acetonitrile and 0.1 formic acid and UV 366 nm, in accordance with embodiments of the present invention.

FIG. 15A depicts structure of naturally driven thiols such as allicin, ajoene (E and Z), sulforaphan, and conjugated sulforaphan, in accordance with embodiments of the present invention.

FIG. 15B depicts structure of naturally driven polyphenols including flavonoids, isoflavone, lignans, stilbenes, and other polyphenols, in accordance with embodiments of the present invention.

FIG. 15C depicts structure of naturally driven punicalagin, ellagic acid, gallagic acid, and other polyphenols, in accordance with embodiments of the present invention.

FIG. 16 is a scheme for sample preparation for LC/MS/MS, in accordance with embodiments of the present invention.

FIG. 17 depicts a LC/MS/MS chromatogram illustrating distinct differences in the detectable levels of Ellagic acid in plasma from animals treated with Nano-Pomegranate extract versus un-detectable levels in plasma from animals treated with plain Pomegranate extracts, in accordance with embodiments of the present invention.

FIG. 18 depicts plasma Levels of ellagic acid in animals treated with plain pomegranate extract versus Nano pomegranate extract, in accordance with embodiments of the present invention.

FIG. 19 depicts procedures for resveratrol extraction in plasma using Solid Phase Extraction, in accordance with embodiments of the present invention.

FIG. 20A depicts a plasma concentration time curve for plain resveratrol versus nano-resveratrol analyzed by LC/MS/MS method, in accordance with embodiments of the present invention.

FIG. 20B depicts resveratrol concentration from oral delivery of nano-resveratrol into the mice liver of mice treated with nano-resveratrol, in accordance with embodiments of the present invention.

FIG. 21 depicts effect of Resveratrol, Z-Ajoene, Resveratrol/Z-Ajoene Nano, in accordance with embodiments of the present invention.

### DETAILED DESCRIPTION

The present invention provides nano co-encapsulating different bioactive compounds derived from different natural products for the prevention and treatment of various disorders via passive and active targeting using naturally derived targeting moieties.

The inventors of the present invention investigated the potential novel nanoformulations in combining polyphenols derived from Ajwa dates, Green tea and Pomegranate with thiols (sulforaphan, ajoene, allicin, and others) derived from garlic and broccoli or cabbage. Naturally driven targeting moieties such as Glycyrrhizin (or glycyrrhizic acid or glycyrrhizinic acid) derived from liquorice were utilized for coating Nano shells for hepatic targeting.

The co-encapsulation in biodegradable sustained release nanoparticles (NPs) may circumvent the preceding limitations. The present invention provides novel formulations of poly (lactic-co-glycolic acid)-Chitosan (PLGA-CH) NPs, Chitosan/tri-methylated chitosan or Fatty acids—Chitosan (FA-CH) NPs loaded with Polyphenols derived from Ajwa shell or seeds and Pomegranate extracts including Punicalagin (PU) or Ellagic acid (EA), wherein CH denotes chitosan. NPs with an average diameter of 150-500 nm were prepared by double emulsion/solvent evaporation method. In one embodiment, chitosan may be derived from plant such as mushroom or crustaceans such as crabs and shrimp.

Pomegranate fruit is a rich source of many polyphenolic compounds with high antioxidant, free radical scavenging, and anti-inflammatory activity, including flavonoids, condensed tannins and hydrolyzable tannins (ellagitannins and gallotanin). Ellagitannins (ETs) are bioactive polyphenols of pomegranates. A predominant ET of pomegranates is punicalagin (PU), and Cathchins. PU is found at high quantities (>2 g/L) in commercial pomegranate juice obtained by squeezing the whole fruit and is responsible for more than half of the total antioxidant capacity of the juice. ETs are prone to spontaneous and enzymatic hydrolysis. For example, PU is hydrolyzed to related ETs, punicalin and gallagic acid, and the final hydrolysis products, ellagic acid (EA) and gallic acid. Pomegranate extract (PE), pomegranate juice and/or individual pomegranate phytochemicals exhibit anticancer effects in vitro and are also bioactive in vivo. PE, containing primarily ETs, exhibits anti-proliferative, pro-apoptotic, anti-invasive and/or anti-inflammatory properties. In addition, PE reduced human prostate and lung cancer xenograft growth. However, poor absorption, low systemic bioavailability and a short retention time of polyphenols such as epigallocatechin-3-gallate (EGCG), ETs and their metabolites may preclude unraveling their full chemo-preventive potential.

Encapsulation of polyphenols such as ETs along with thiols such as sulforaphan into biocompatible and biodegradable NPs may overcome susceptibility of thiols to gastrointestinal instability, poor absorption, low systemic bioavailability and a short half-life in addition to improved efficacy and safety when administered alone or with commonly used pharmaceuticals.

Studies conducted by the inventors of the present invention demonstrate that a nano-composition comprising co-encapsulation of polyphenols such as epigallocatechin-3-gallate (EGCG) from green tea, resveratrol from grapes, curcumin from turmeric with Thiols (sulforaphan, ajoene, allicin, and others) from aged garlic, Indole from broccoli or cabbage are highly synergistic with improved pharmacokinetic and pharmacodynamic profiles as compared to their free counterparts which are not co-encapsulated. Poly (lactic-co-glycolic acid)—Chitosan (PLGA-CH) NPs, Chitosan/tri-methylated chitosan NPs, and/or FA-CH NPs are biocompatible, biodegradable and stable in biological fluids, protect the loaded compounds from degradation and provide sustained release of the loaded compounds. Additionally, targeting moieties such as Glycyrrhizin derived from liquorice were utilized for coating Nano shells for targeting particular locations (e.g., hepatic targeting) within the subject using one or more targeting moieties coated on nanoparticles of the nano-composition.

The nano-composition of the present composition may be utilized by a subject (e.g., a mammal such as a human being) via oral, topical, or other routes of delivery.

The nano-composition may be utilized for cancer applications, by inhibiting angiogenesis, by providing anti-cancer and enhanced chemotherapy/radiotherapy efficacy, while improving the safety of chemotherapy/radiotherapy and wherein safety was demonstrated with polyphenols/thiols with various nano-shells.

The nano-composition may be utilized for suppression of tissue fibrosis. Anti-fibrotic efficacy of Nano-encapsulated polyphenols/thiols with various nano-shells coated with Glycyrrhizin was demonstrated in various tissue fibrosis such as hepatic, pulmonary, cardiac, kidney fibrosis, and fibrosis-associated disorders such as scleroderma, and others fibrotic disorders. Synergistic and sustained maximal anti-fibrotic effects were achieved with co-encapsulate polyphenols and thiols in a nano shell along with targeting moiety conjugated to the outside surface of the nano shell.

The nano-composition may be utilized for treatment of inflammatory disorders. Nano-encapsulated polyphenols/thiols with various nano-shells are effective in inflammatory bowel diseases, rheumatoid arthritis, osteoarthritis, and other acute and chronic inflammatory disorders.

The nano-composition may be utilized for treatment of infectious diseases. Ajwa extracts as well as nano-encapsulated polyphenols/thiols with various nano-shells are effective against infection. The scope of an “Ajwa extract” includes both the meat and the seeds of an ajwa date.

### Example 1

### Date Ajwa Flesh and Seed Extraction: Method 1: 70% Ethanol Extraction

Date flesh (50 g) was cut to small piece and moved into two 50 ml conical centrifuge tubes, and 35 ml of ethanol and water (7:3) was added to each tube to form a mixture. The mixture was homogenized and vortexed for 15 min. After gently stirring the mixture at room temperature over 20 hours, the mixture was centrifuged to eliminate the precipitate. The upper brown clear solution was removed into another conical centrifuge tube to lyophilize. It is difficult to get dry powder because the mixture contains high level of fructose. After concentration through lyophilization, total of 22 ml of viscous brown liquid was obtained from 50 g of date flesh.

Date seed (16.5 g) was smashed in the presence of liquid nitrogen. The extraction procedure is same as the preceding date flesh extraction with 70% ethanol. Finally, 2.5 ml of viscous brown liquid was obtained from 16.5 g of date seed.

### Example 2

### Date Ajwa Flesh and Seed Extraction: Method 2: Solvent Extraction

Date flesh (50 g) was cut to small piece and moved into two 50 ml conical centrifuge tubes, and 35 ml of deionized water was added to each tube to form a mixture. The mixture was homogenized. The resulted mixture of date flesh homogenate was removed into a 250 ml glass bottle, and 150 ml of tBME (tert-Butyl methyl ether) was added. The mixture was gently stirred at room temperature overnight, and then centrifuged. The upper solvent phase was removed into another glass bottle, dried under gentle nitrogen stream, and its residual was resuspended with 1.5 ml of Ethyl acetate. The water phase was removed into a 50 ml conical centrifuge tube to be lyophilized. It is difficult to get dry powder because the mixture contains high level of fructose. After concentration through lyophilization, total of 30 ml of viscous brown liquid was obtained from 50 g of date flesh.

### Example 3

### Extraction and Analysis of Date Seed Extract Using Chromatography

Instrument and Chemicals: HPLC: Waters 2695 Alliance Separations Module with Column Oven and 2996 Photo Diode Array Detector; LC-MS system: API 4000 bench top triple quadrupole interfaced two Shimadzu 20-AD pumps, a SIC-20AC auto sampler and a CTO-20AC column oven; Analytical column: Phenomenex, Luna NH2, 5μ, 4.6×250 mm; Solid phase extraction (SEP) cartridge: Spe-ed™ Amberlite XAD-7 resin); Freeze Dry Systems: LABCONCO Free Zone 6; Filter: VWR vacuum filtration system, nylon, 0.2 μm, 500 ml; and HPLC grade methanol and acetonitrile (Fisher Scientific); formic acid ˜98%, (Fluka); super purified water (Millipore).

### Example 4

### Powder of Roasted Date Seed

Extraction and separation of roasted date seed. Some studies (conducted in China, unpublished data) indicated that the potential effective composition in date seed is the flavonoids, which have shown bioactivities such as anti-oxidation, anti-cancer and vaso-protection. This first step of this study is to extract the composition containing flavonoids from the date seed.

15 g of roasted date seed powder was weighed and marinated in 300 ml of 60% ethanol at 80° C. for 3 hours (stirred occasionally). The ethanol extract, after being cooled, was filtrated through 0.2 μm nylon membrane by vacuum. An aliquot of filtrate was diluted 1:20 (v: v) with methanol and applied onto mass spectrometry scan. The rest of filtrate was dried with vacuum evaporator for further separation and normal HPLC analysis.

### Example 5

### HPLC UV Scan of Date Seed Extract

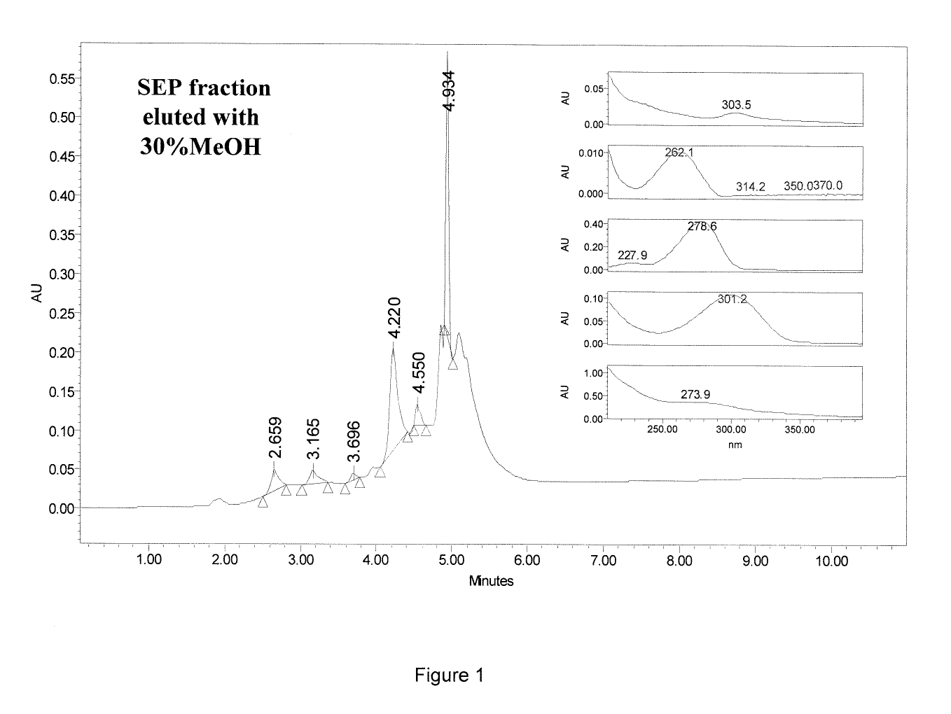

An aliquot of dried date seed extract was dissolved with 25% methanol and loaded onto SEP cartridges (Amberlite XAD-7), which were eluted with 30, 45, 60, 70, and 100% methanol and the elutes were collected, respectively. Each of the fractions was loaded onto a HPLC column (Luna, NH2), eluted in gradient with 98-90% acetonitrile containing 0.1% formic acid, and detected by UV (190-410 nm) (see FIGS. 1-5).

FIG. 1 depicts SEP Fraction eluted with 30% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid (UV 254 nm).

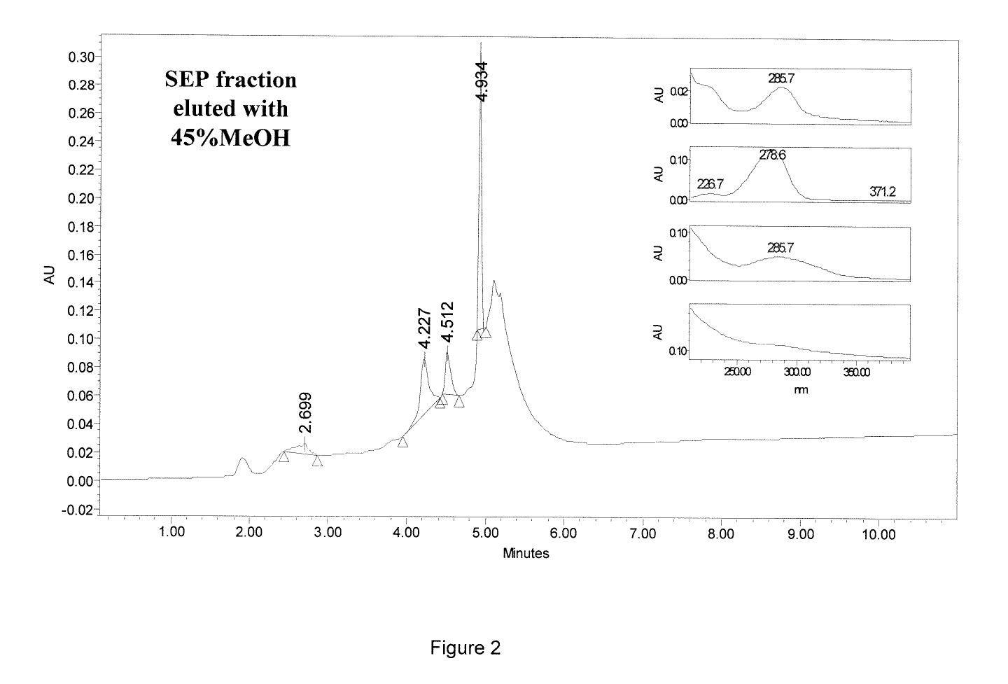

FIG. 2 depicts SEP Fraction eluted with 45% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid (UV 254 nm).

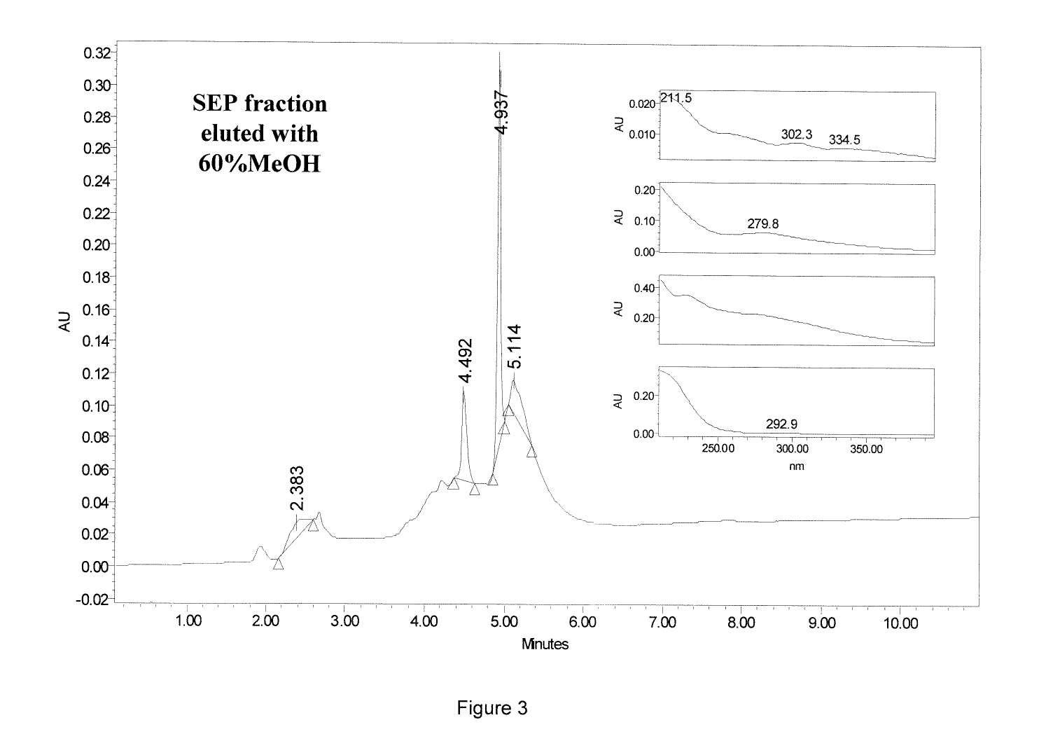

FIG. 3 depicts SEP Fraction eluted with 60% Methanol—HPLC chromatogram and UV spectrum of date seed extract fraction through XAD-7 resin, in accordance with embodiments of the present invention. NH2 column eluted in gradient with 98-90° % methanol containing 4 mM ammonium acetate and 0.1% formic acid (UV 254 nm).

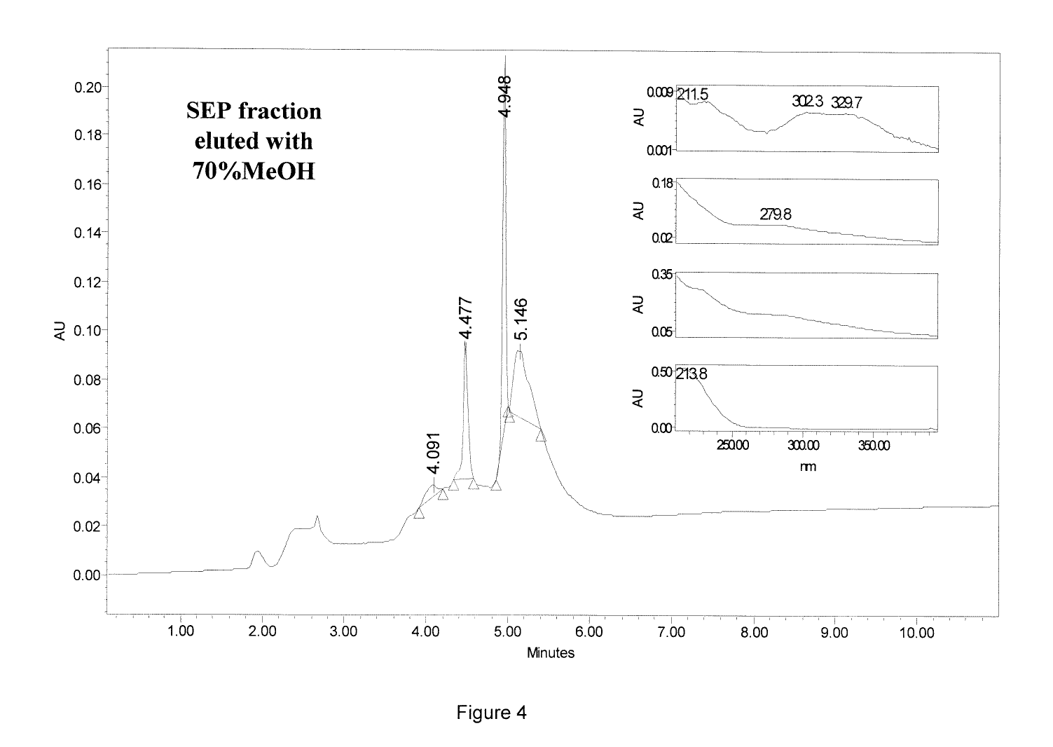

FIG. 4 depicts SEP Fraction eluted with 70% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid (UV 254 nm).

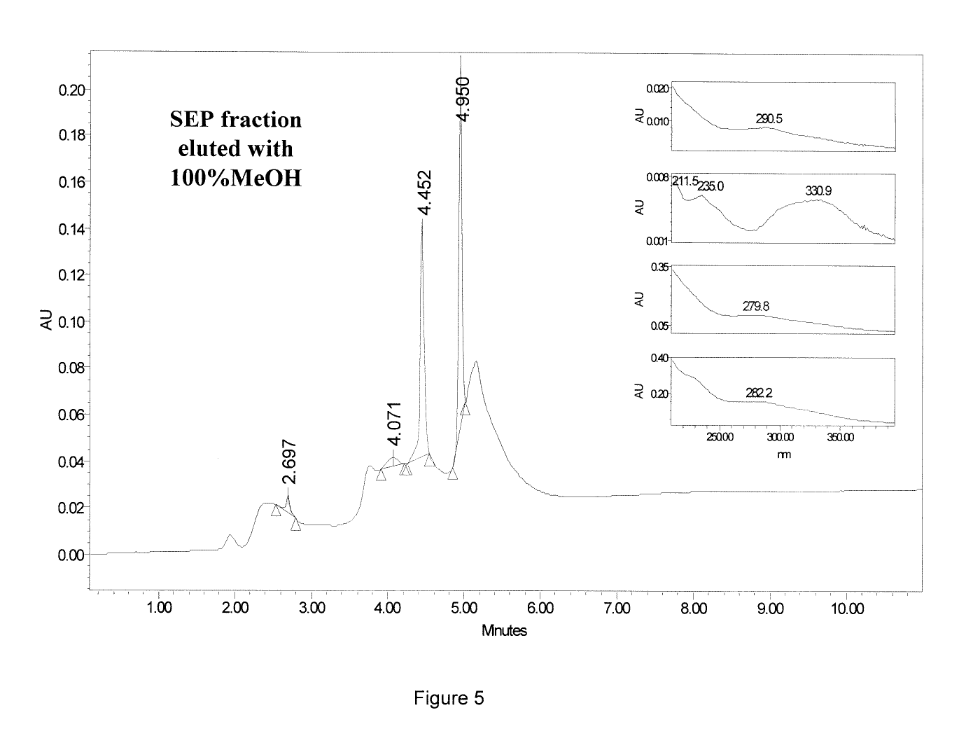

FIG. 5 depicts SEP Fraction eluted with 100% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid (UV 254 nm).

### Example 6

### MS Scan and LC-MS Analysis of Date Seed Extract

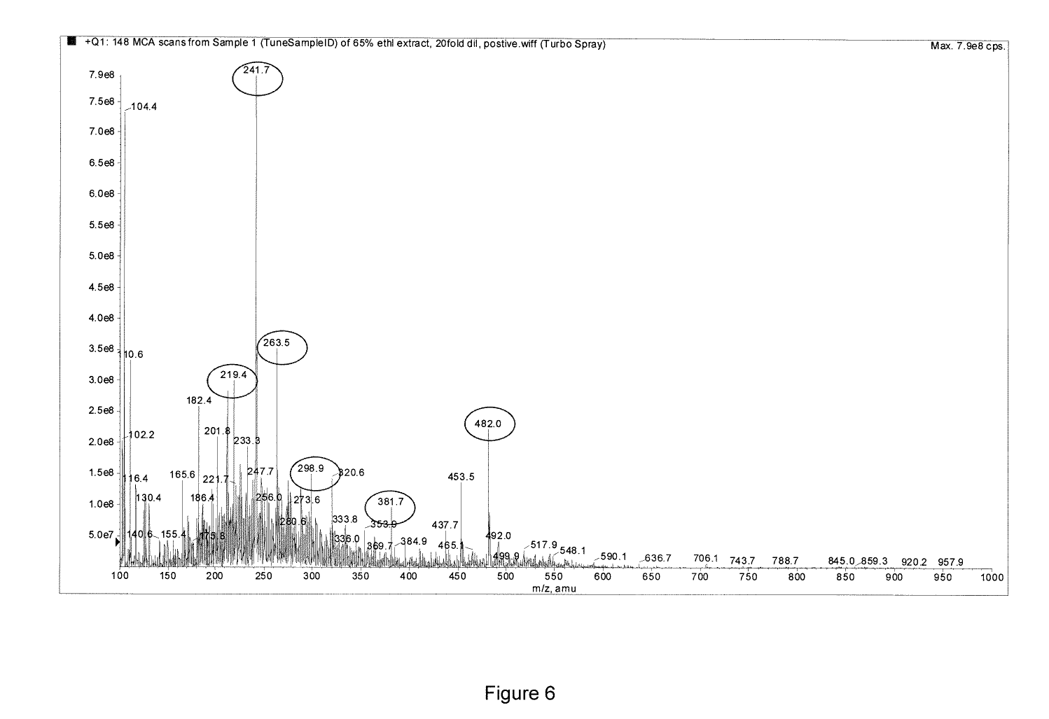

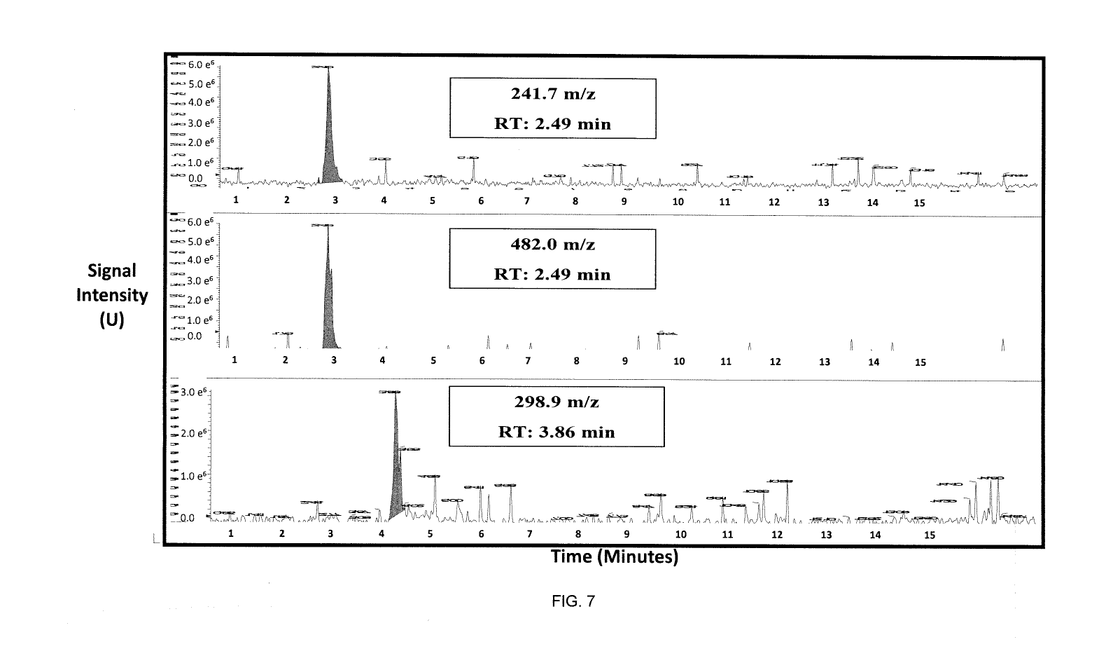

The diluted extract (1:20) was scanned with mass spectrometry from 100 through 1000 m/z. Compared with the mass spectrogram of the control matrix (95% methanol), several ion peaks were only appeared in the extract of date seed (see FIG. 6), which strongly suggested that these components are specific to date seed. The same diluted extract was further separated onto a normal phase HPLC column (Luna, NH2), by a gradient elution with 98-90% methanol containing 4 mM ammonium acetate and 0.1% formic acid, and detected by LC-MS under selected ion monitoring (SIM) model. Among the acquired SIM plots, three peaks corresponded with the unique masses found by MS scan (see FIG. 7).

FIG. 6 depicts a mass spectrogram of 60%°, ethanol extract of date seed, in accordance with embodiments of the present invention. MS scanned from 100 through 1000 m/z in positive Q1 model. Compared with the control matrix (95% methanol), these ion peaks (circled) only appear in the 60% ethanol extract of date seed powder.

FIG. 7 depicts SIM (selected ion monitoring) plots of 60% ethanol extract of date seed powder, in accordance with embodiments of the present invention. LC conditions: NH2 column eluted in gradient with 98-90% methanol containing 4 mM ammonium acetate and 0.1% formic acid. Three peaks corresponded with the unique masses found by MS scan.

### Example 7

### Synthesis of Nanoparticles

PLGA-CH or FA-CH nanoparticles co-encapsulating polyphenols and thiols (sulforaphan, ajoene, allicin, and others) were prepared by double emulsion/solvent evaporation methods. A stock solution of PLGA-CH or FA-CH polymer was prepared by dispersing of polymers (PLGA/CH or FA/CH ratio of 2-3/1) in ethyl acetate. A stock solution of 10 mg/ml polyphenols/thiols (sulforaphan, ajoene, allicin, and others) was prepared by dissolving in ethyl acetate. Five hundred μl of each stock solution was mixed together by vortexing. Then, 1 ml of this solution, containing of 40 mg/ml PLGA-CH or FA-CH and 5 mg/ml polyphenols/thiols (sulforaphan, ajoene, allicin, and others) was mixed with 200 μl of PBS by intermittent sonication (2-3 times, 30 sec each time) to obtain primary emulsion. The primary emulsion was then intermittently emulsified by sonication (30 s) in 2 ml of 1% w/v PVA solution. This water-in-oil-in-water emulsion was then added to 40 ml of 1.0% PVA solution and stirred for 30 min under constant magnetic stirring. Immediately after, ethyl acetate was evaporated at low pressure at 37° C. using a rotatory evaporator. The whole solution was then dialyzed using 10-12 KD dialysis membrane against water for 24 hours to remove non-encapsulated polyphenol, thiols (sulforaphan, ajoene, allicin, and others). The entire solution was lyophilized and re-dispersed for further use.

### Example 8

### Self-Assembly of Polyphenol (Green Tea Catechin Derivatives) Nanoparticles

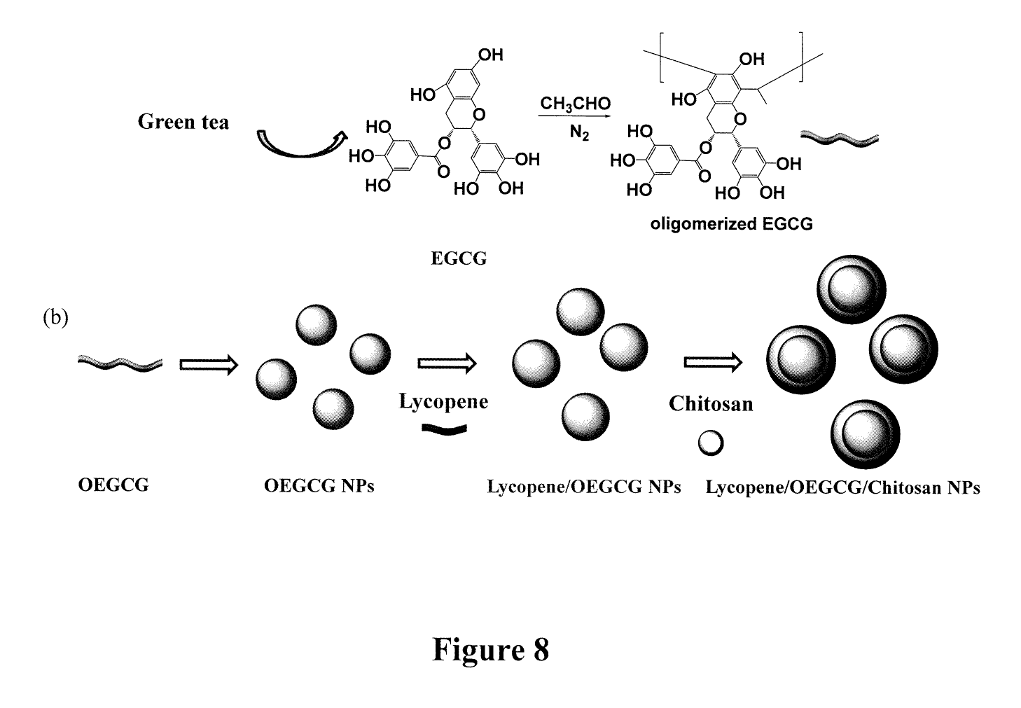

A self-assembly process is used to form the Lycopene/OEGCG/Chitosan or Methylated Chitosan NPs, which are formed via two sequential self-assemblies processes in an aqueous solution: complexation of OEGCG with lycopene to form the core, followed by coating with chitosan to form the shell (see FIG. 8). OEGCG denotes oligomer EGCG.

### Example 9

### Self-Assembly of Polyphenol (Green Tea Catechin Derivatives) Nanoparticles

A self-assembly process is used to form the Lycopene/OEGCG/Chitosan or methylated Chitosan NPs, which are formed via two sequential self-assemblies processes in an aqueous solution: complexation of OEGCG with lycopene to form the core, followed by coating with chitosan to form the shell. Freeze drying of this nanoparticle was also evaluated as a means to improve shelf life. Then, formulations were administered by oral gavage into mice.

### Example 10

Freeze-dried chitosan nanoparticles (CS NPs) were prepared. The prepared Chitosan-coated NPs can prevent the release of EGCG from CS NPs in the stomach and enhance absorption of the CS NPs on the surface of the small intestine, thus further increasing bioavailability of EGCG. Freeze drying of this nanoparticle was also evaluated as a means to improve shelf life. Then, formulations were administered by oral gavage into mice. CS NPs, chitosan nanoparticles.

### Example 11

### Nanoformulation (I) for Water Soluble Extract or its Bioactive Compounds

Complex formation of Chitosan derivative and Hyaluronic acid derivative for encapsulation of water soluble extract or its bioactive compounds may be implemented.

### Example 12

### Nanoformulation (II) for Water Soluble or Insoluble Extract and its Bioactive Compounds

Complex formation of chitosan or tri-methyl chitosan and oleic acid, myristic acid, hyaluronic acid, and caprylic acid for encapsulation of water soluble or insoluble extract and its bioactive compounds may be implemented wherein the percent by weight of Chitosan or tri-methyl Chitosan (Average Molecular weight 10-30 KD) in the nanoparticle shell ranged from greater than 10% to less than 500/%, and Oleic acid, Myristic acid, Hyaluronic acid, and Caprylic acid in the nanoparticle shell ranged from greater than 50% to less than 90%.

### Example 13

### Size Measurement by Dynamic Light Scattering

Size distribution of the NPs in aqueous dispersion was determined by dynamic light scattering (DLS) using a Malvern zeta sizer (Malvern Instrumentation Co, USA). After the re-dispersion of the lyophilized powder in deionized water, 1 ml of the NP solution was taken in 3 ml of a four size clear plastic cuvette and measured directly by the DLS. Nano composite average sizes ranged from 150-500 nm, with zeta potential +10 to +30 mv and −10 to −30 mv.

### Example 14

### Procedures for Preparation of Dates Extract

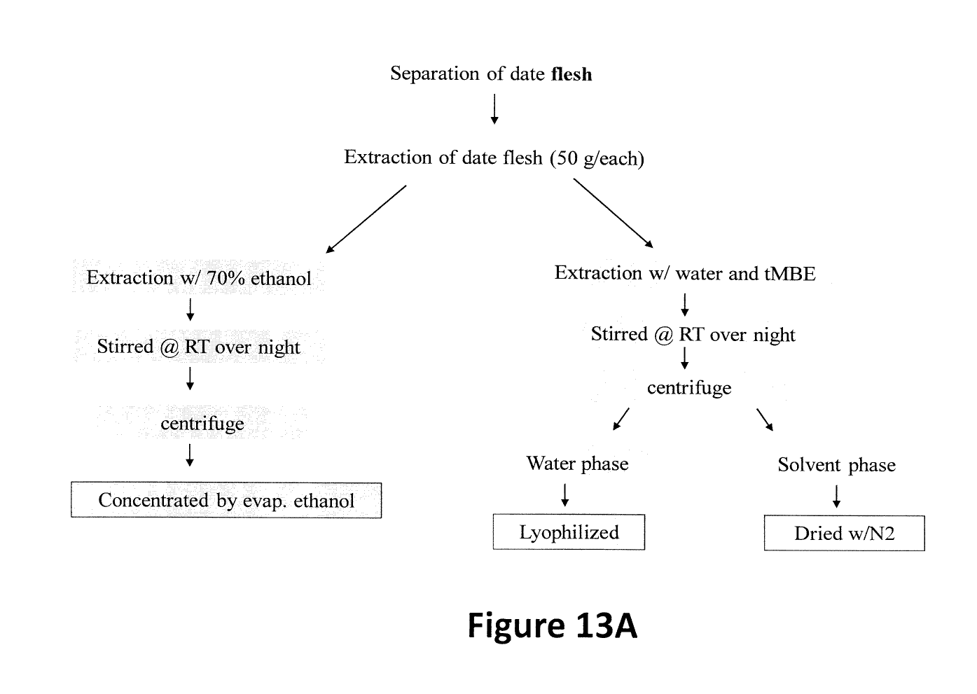

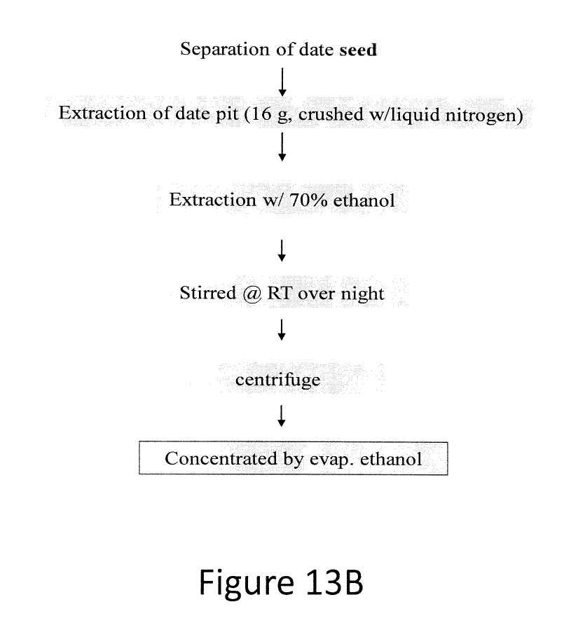

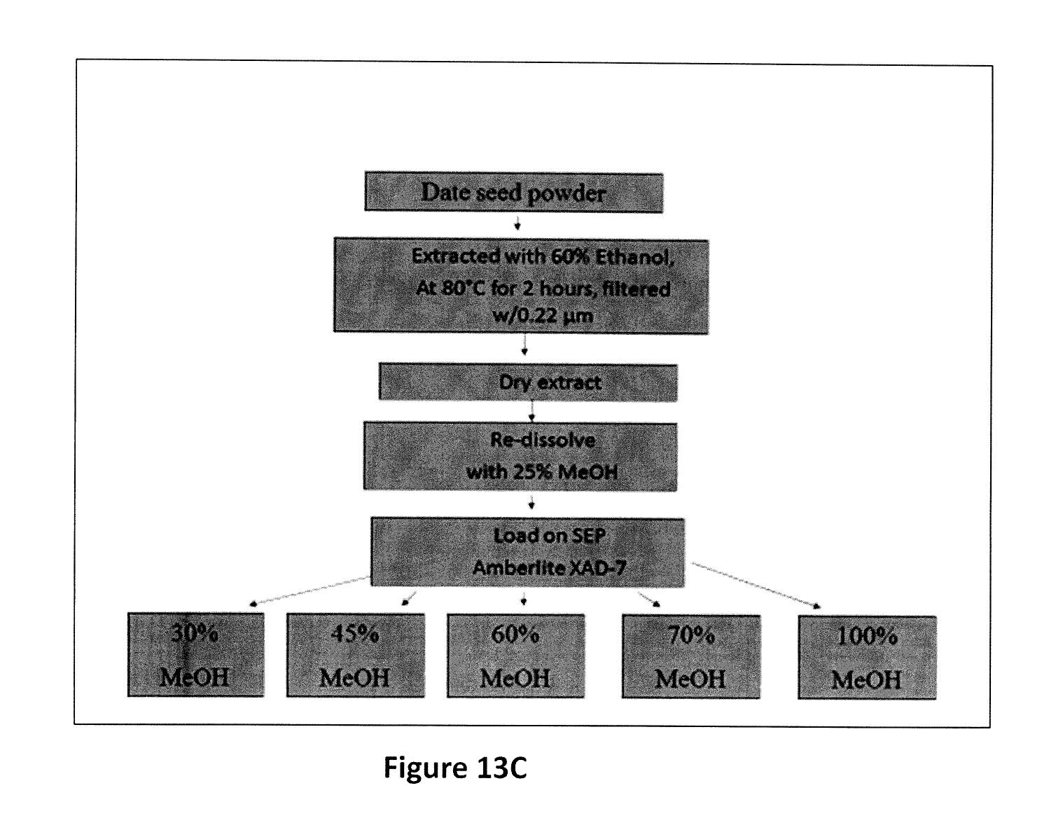

Extraction of dates flesh or seed was carried out using aqueous solution for water soluble bioactive ingredients and with 70% ethanol for hydrophilic and hydrophobic ingredients (see FIGS. 13A and 13B). Extracts were further concentrated and dried down for further evaluations. Additionally, date extracts were purified using solid phase columns (see FIG. 13C).

### Example 15

### Mass Spectroscopic Analysis of Date Extract

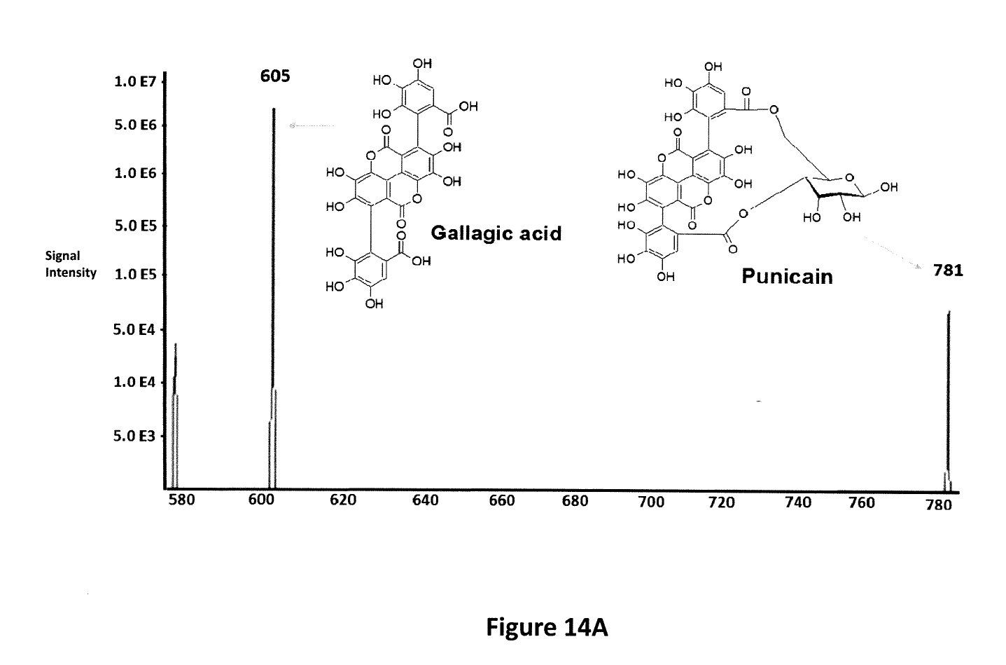

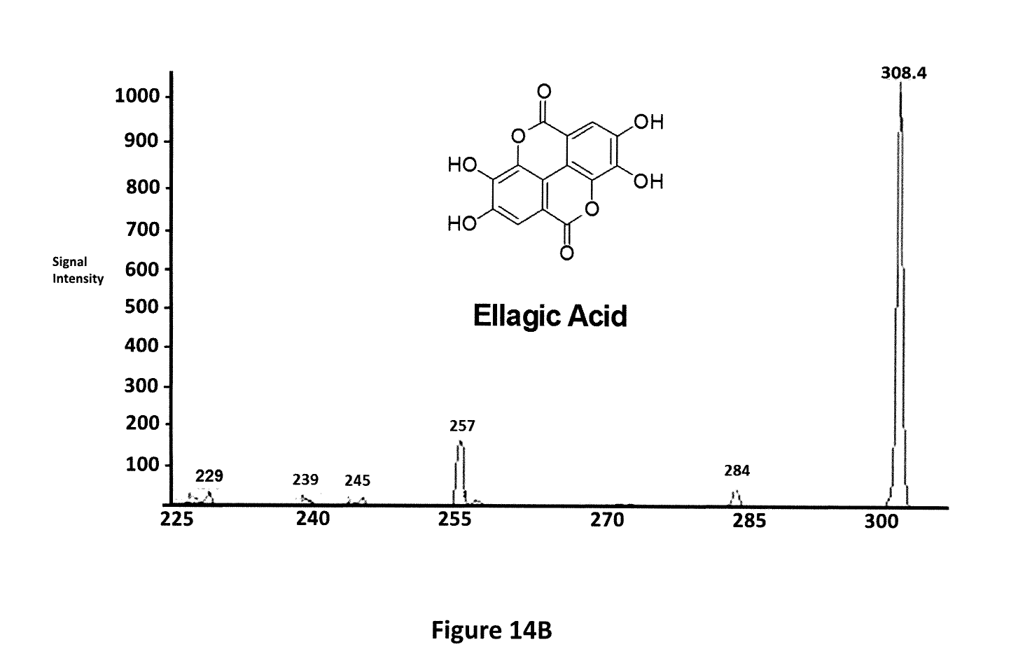

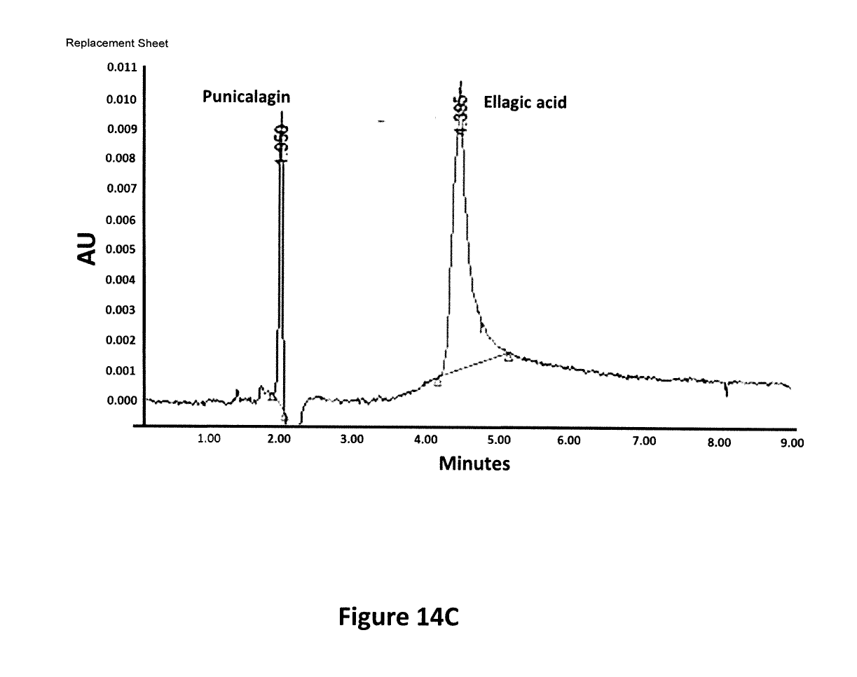

Ellagic acid. Gallagic acid, and Punicalin were extracted in date extracts (see FIGS. 14A and 14B). Analysis of the levels of punicalagin and ellagic acid was carried out using LC/MS/MS analysis (see FIG. 14C).

### Example 16

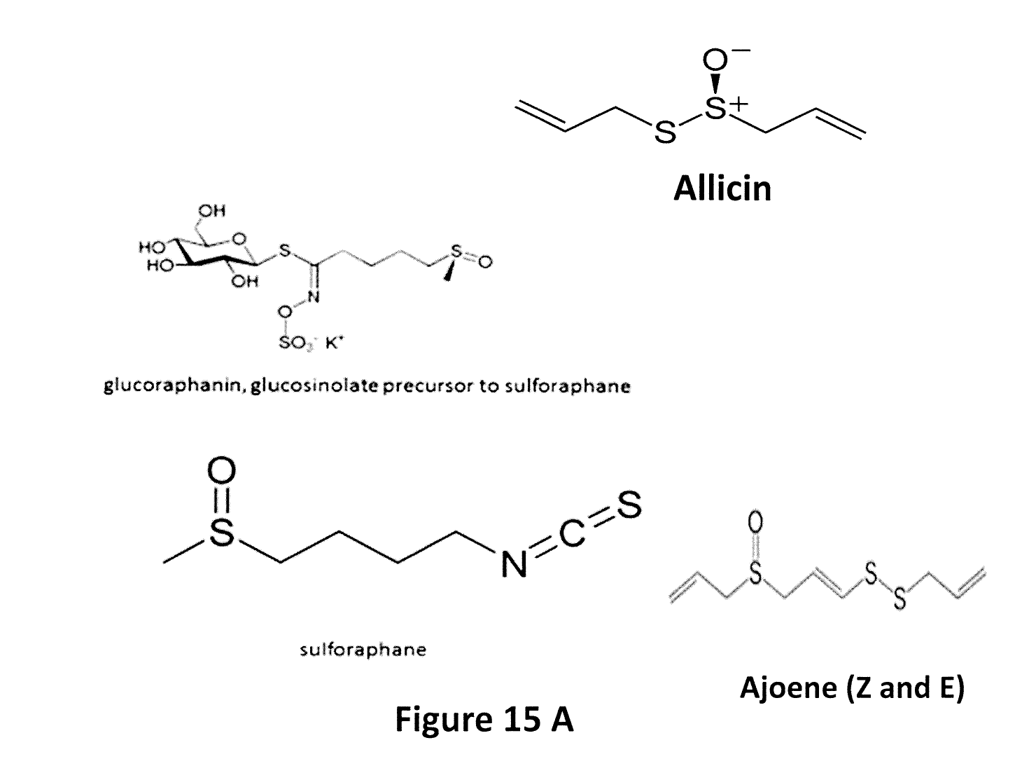

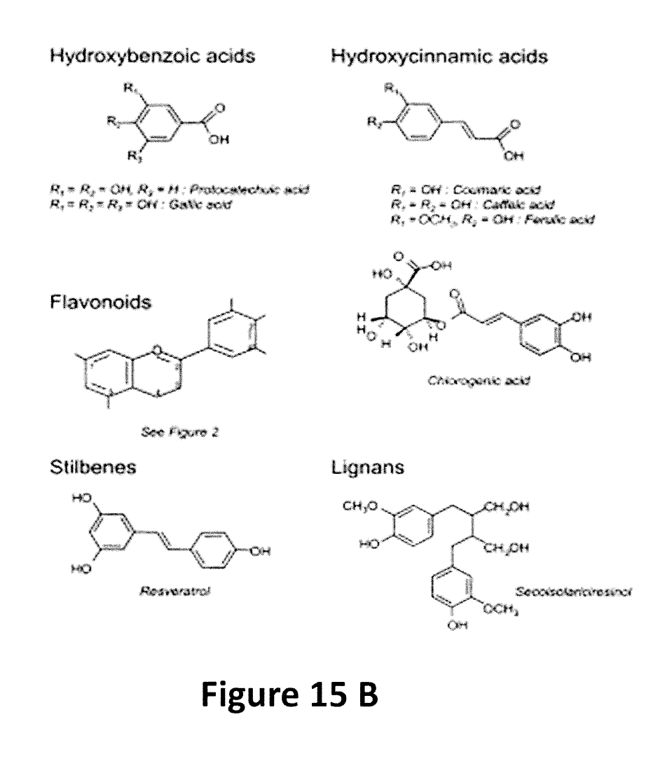

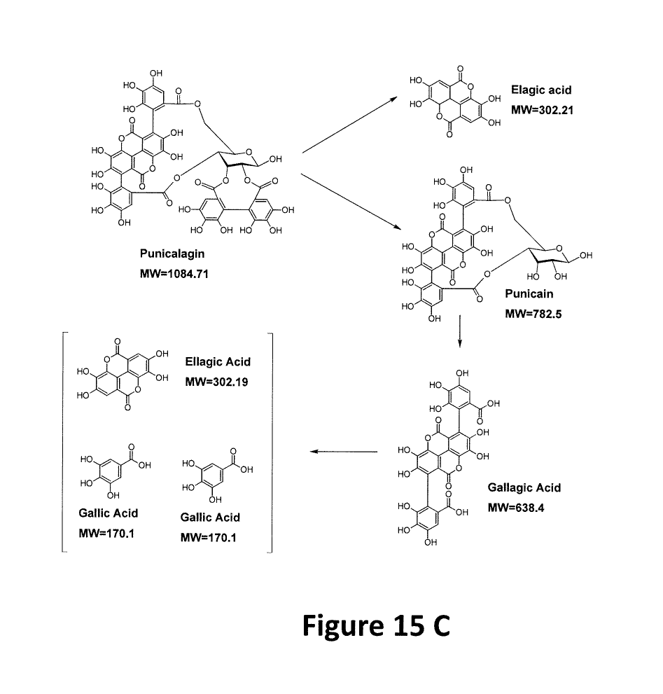

Thiols extracted from natural products (FIG. 15A), Polyphenols extracted from Ajwa and Pomegranate extract (FIG. 15 B, C).

### Example 17

### Procedures for Sample Preparation for LC/MS/MS

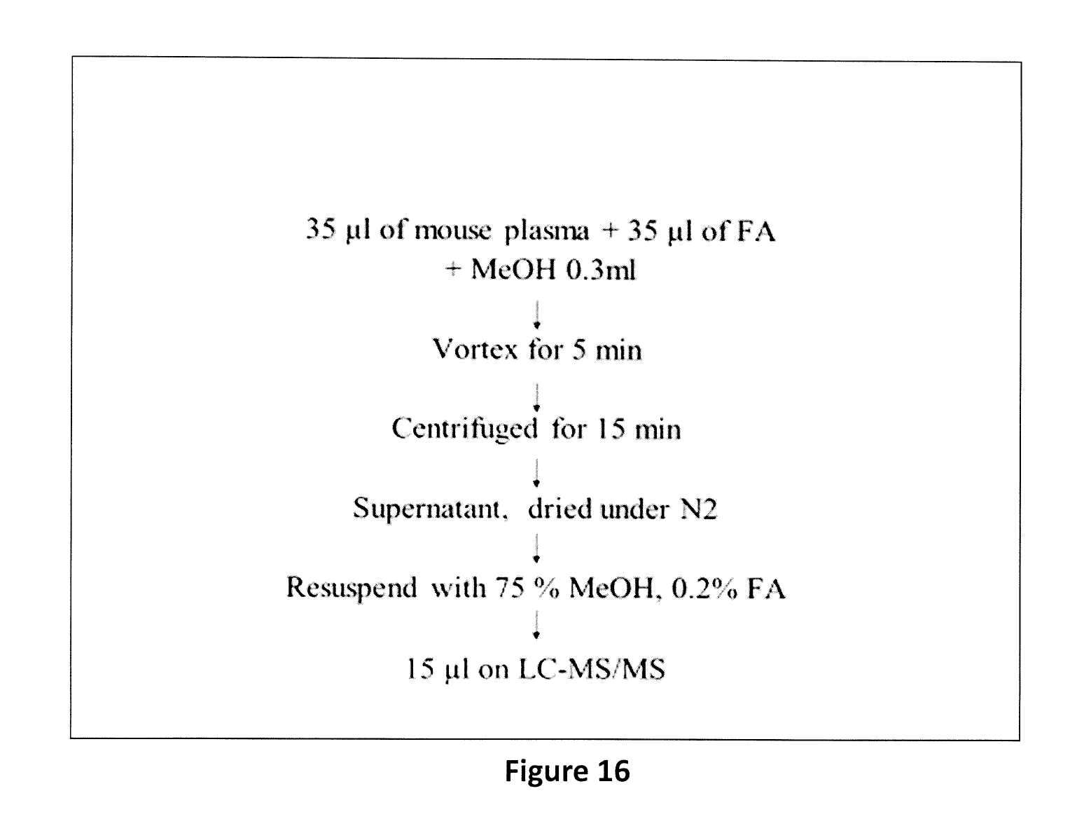

Mouse plasma was mixed with formic acid (FA) and methanol (MeOH), vortexed, centrifuged and supernatant was dried under Nitrogen. The dried supernatant was re-suspended with 75% methanol and 0.2% FA and the sample was injected into the LC/MS/MS (see FIG. 16).

### Example 18

### Animals

Mice n=4 per group, compound to be tested: Plain pomegranate extract versus Nano pomegranate extract, Dose: 1 mg/Kg (equivalent 0.03 mg Ellagic acid mg/Kg), Oral, Blood samples at different intervals for LC/MS/MS analysis.

### Example 19

### PK Study of Pomegranate Extract in Mice

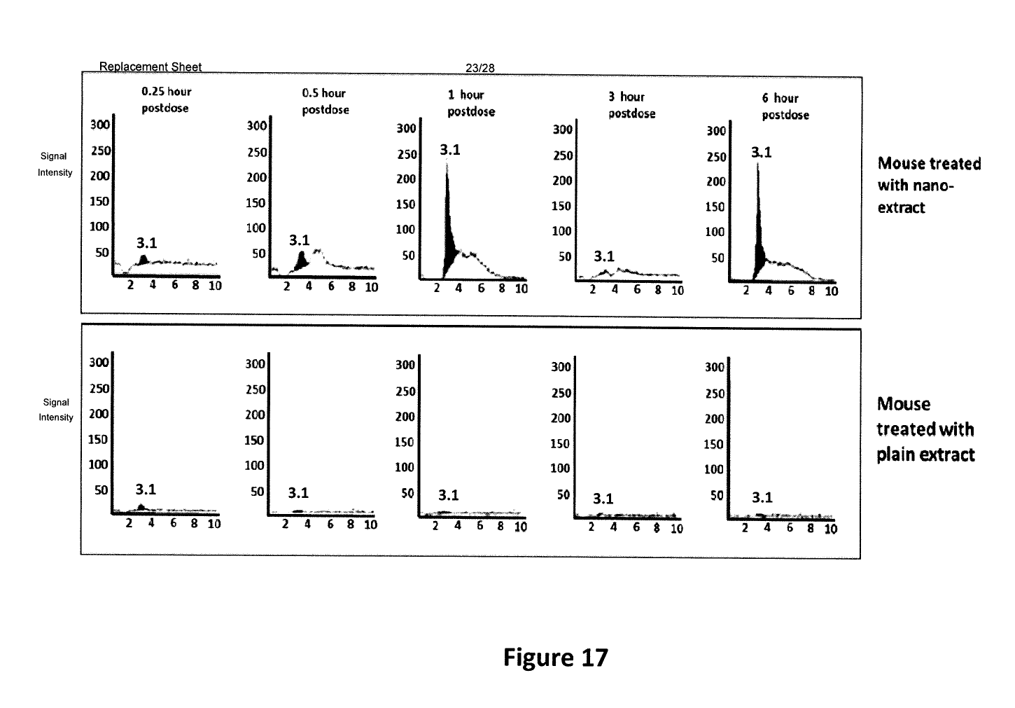

LC/MS/MS chromatogram illustrated a distinct differences in the detectable levels of Ellagic acid in plasma from animals treated with Nano-Pomegranate extract versus un-detectable levels in plasma from animals treated with plain Pomegranate extracts (see FIG. 17).

### Example 20

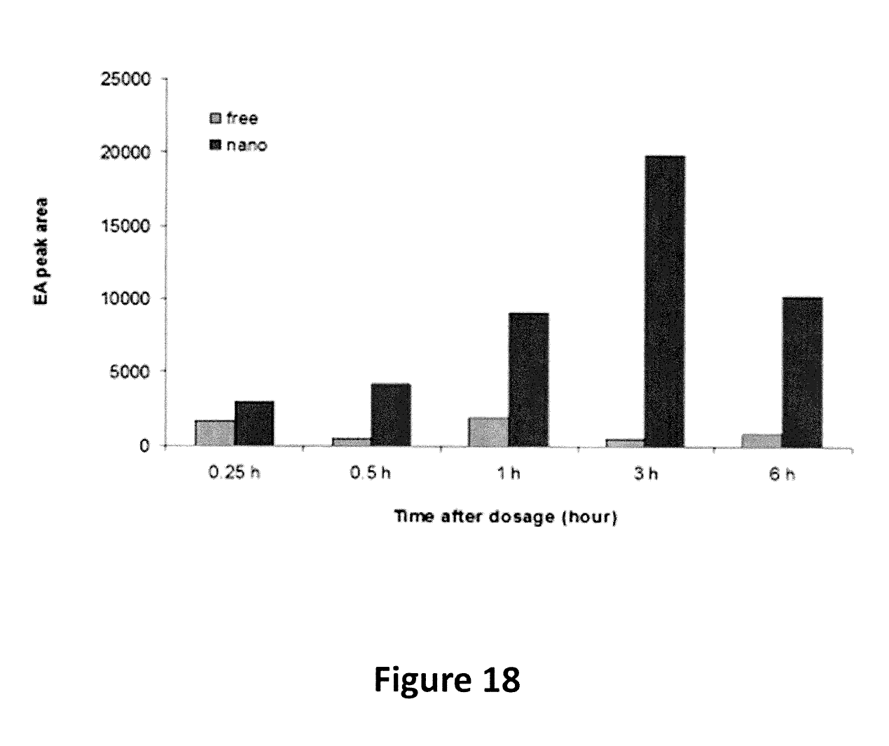

FIG. 18 shows plasma Levels of ellagic acid in animals treated with plain pomegranate extract versus Nano pomegranate extract. The data shows distinct differences in the detectable levels of Ellagic acid in plasma from animals treated with Nano-Pomegranate extract (Area Under the Curve, AUC ranged from 2,000-20,000) versus very low to un-detectable levels (AUC ranged from 200 to 1,000) in plasma from animals treated with plain Pomegranate extracts as shown in FIG. 18.

### Example 21

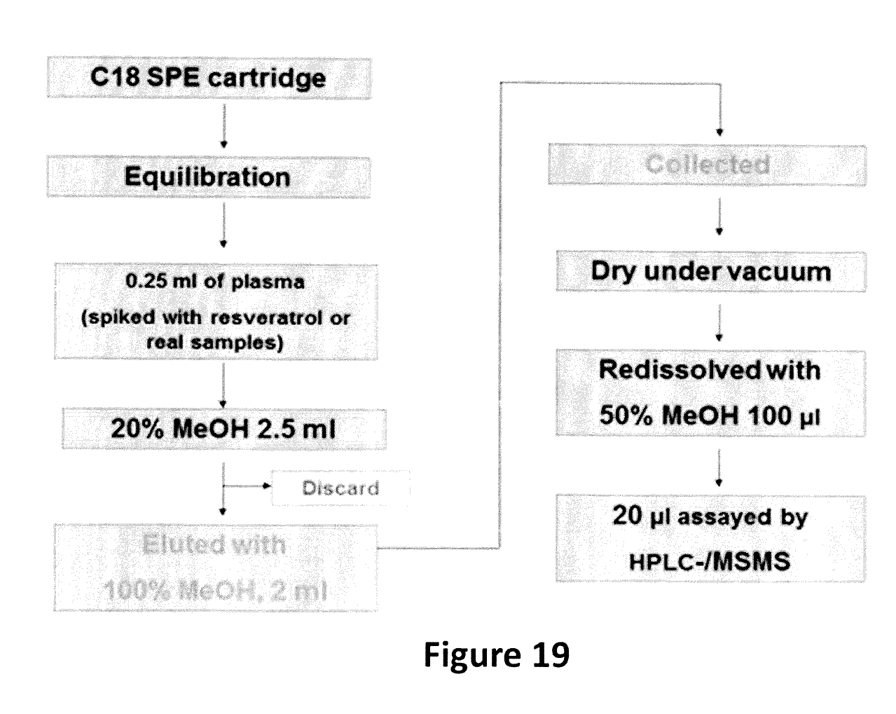

Animals (Mice) treated with plain resveratrol versus nano-resveratrol at 4 mg/kg, by subcutaneous was studied. Blood samples were taken into heparinized capillaries at 5, 15, 30, 60, 180, and 360 minutes post-dosing. Extraction of resveratrol spiked in plasma versus plasma from treated animals was carried out as shown in FIG. 19.

### Example 22

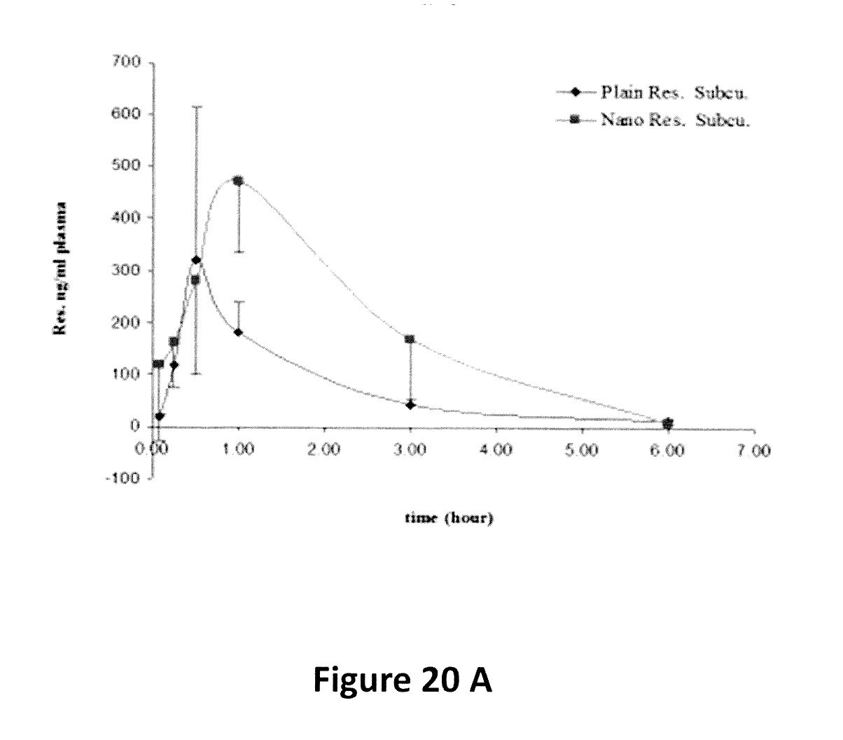

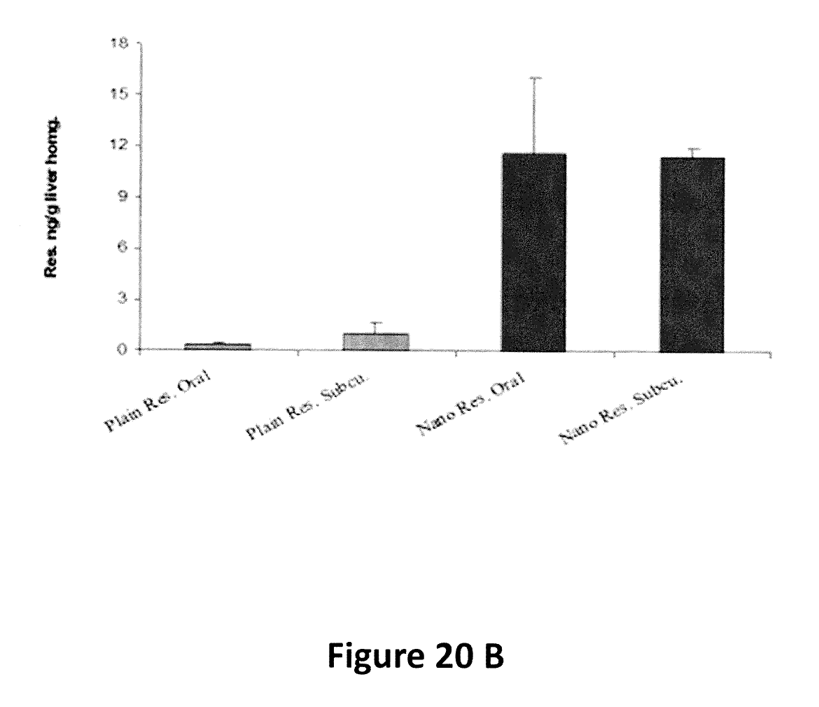

Plasma concentration time curve for plain resveratrol versus nano-resveratrol analyzed by LC/MS/MS method, Data showed greater levels in plasma from animals treated with nano-resveratrol versus those treated with plain resveratrol subcutaneously (FIG. 20A) Oral delivery into the mice liver of mice treated with nano-resveratrol was much greater as compared to those animals treated with plain resveratrol after either subcutaneous or oral administration (FIG. 20B).

### Example 23

### Cancer Cell Lines and Reagents

Human pancreatic cancer cell lines, MPanc96 expressing firefly luciferase, were provided by MD Anderson Cancer Center, Houston, Tex. Cell culture reagents and hemoglobin standard, Drabkin's reagent, Ellagic acid, DIM and other common reagents were purchased from Sigma (St. Louis, Mo.). D-Luciferin potassium salt was purchased from Caliper Life Sciences (Hopkinton, Mass.). Matrigel was purchased from BD Bioscience (San Jose, Calif.).

Cells and Cell Culture: Mpanc96-luc cells were grown in DMEM supplemented with 5% fetal bovine serum, 1% penicillin, and 1% streptomycin. Cells were cultured at 37° C. to sub-confluence and treated with 0.25% (w/v) trypsin/EDTA to affect cell release from culture flask. After washing cells with culture medium, cells were suspended in DMEM (free of phenol red and fetal bovine serum) and counted.

### Example 24

### Synthesis of Nanoparticles

Different Nanoformulations: Polyphenols such as Ellagic acid (EA), Resveratrol, and Thiol (sulforaphan, ajoene, allicin, and others) containing compounds derived from natural products such as alkyl sulfhydryl compounds or their combination were encapsulated into PLGA-CH-PEG nanoparticles by double emulsion/solvent evaporation methods as previously described. Briefly, a stock solution of PLGA-CH-PEG polymer was prepared by dispersing 80 mg/ml of this polymer in dichloromethane. A stock solution of 10 mg/ml of was prepared by dissolving the different naturally derived polyphenols or thiol (such as sulforaphan, ajoene, allicin, and others) in dichloromethane. Five hundred μl of each stock solution was mixed together by vortexing. Then, 1 ml of this solution, containing of 40 mg/ml PEG-PLGA and 5 mg/ml polyphenols or alkyl sulfhydryl was mixed with 200 μl of PBS by intermittent sonication (2-3 times, 30 sec each time) to obtain primary emulsion. The primary emulsion was then intermittently emulsified by sonication (30 s) in 2 ml of 1% w/v PVA solution. This water-in-oil-in-water emulsion was then added to 40 ml of 1.0% PVA solution and stirred for 30 min under constant magnetic stirring. Immediately after, dichloromethane was evaporated at low pressure at 37° C. using a rotatory evaporator. The whole solution was then dialyzed using 10-12 KD dialysis membrane against water for 8 h to remove the impurities. The entire solution was lyophilized and re-dispersed for further use.

### Example 25

### Size Measurement by Dynamic Light Scattering

Size distribution of the different Nanoformulations Ellagic acid (EA), Resveratrol, Polyphenols, Thiol sulforaphan, ajoene, allicin, and others) containing compounds derived from natural products or their combination in aqueous dispersion was determined by dynamic light scattering (DLS) using a Malvern zeta sizer (Malvern Instrumentation Co, USA). After the re-dispersion of the lyophilized powder in deionized water, 1 ml of the NP solution was taken in 3 ml of a four size clear plastic cuvette and measured directly by the DLS.

### Example 26

### MTT Cell Viability Assay

MPanc96 cells were subjected to treatment with combinations, their Nanoformulation at concentrations ranging from 0.1 to 10 μg. After 24, 48, and 72 h of exposure to the compounds, cell viability was determined by MTT viability assay, as per the manufacturer's protocol. Briefly, cells were seeded at a density of 10⁴ cells/well in 96-well plates, and then incubated with compounds. After treatment, MTT solution was added to each well, and plates were incubated for 4 h at 37° C. The cells were solubilized by the addition of 50 μl of DMSO and incubated for 10 min at 37° C. The optical density of each well was determined using an ELISA plate reader at an activation wavelength of 570 nm and reference wavelength of 650 nm. The percentage of viable cells was determined by comparison to untreated control cells.

### Example 27

### Effect of Thiol (Z-Ajoene), Polyphenol (Resveratrol) and their Combination on Angiogenesis

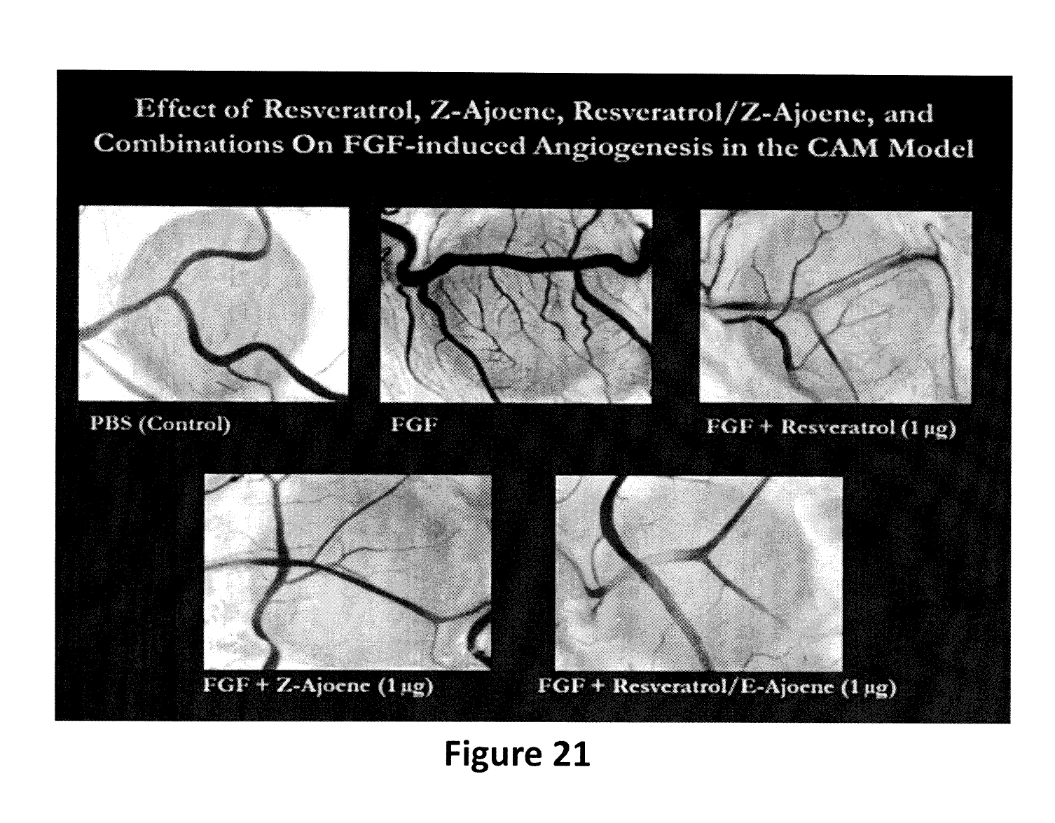

Synergistic anti-angiogenesis was shown when Z-ajoene and resveratrol were combined as shown in FIG. 21.

### Example 28

### Tumor Growth in the CAM Cancer Implant Model

To determine the relative potency of different Nanoformulations such as Ellagic acid (EA), Resveratrol, Polyphenols, Thiol (sulforaphan, ajoene, allicin, and others) containing compounds derived from natural products such as or their combination versus Free EA, Resveratrol, Polyphenols, Thiols sulforaphan, ajoene, allicin, and others) or combinations thereof in the CAM pancreatic cancer cell implant model of tumor growth and tumor angiogenesis.

A 7-day old chick embryo was purchased from Spafas, Inc. (Preston, Conn.) and incubated at 37° C. with 55% relative humidity. A hypodermic needle was used to make a small hole in the shell at the air sac and a second hole will be made on the broadside of the egg, directly over an avascular portion of the embryonic membrane that was identified by candling. A false air sac was created beneath the second hole by the application of negative pressure at the first hole, causing the CAM to separate from the shell. A window, approximately 1.0 cm², was cut in the shell over the dropped CAM with a small crafts grinding wheel (Dremel, Division of Emerson Electric Co., Racine, Wis.), allowing direct access to the underlying CAM. Briefly, MPanc96-Luc C6 cells were implanted at 1 million cells/CAM in Matrigel at the 7-day old fertilized chick egg. Treatment effects (Tumor growth, viable cancer cells, and tumor angiogenesis) were determined 7 days after tumor cell implantation. For these studies, Matrigel® (BD Bioscience, San Jose Calif.) will be thawed overnight at 4° C. and placed on ice. Cells in exponential growth phase will be harvested using 0.25% trypsin-EDTA washed and suspended in medium. Only suspensions of single cells with a viability exceeding 95% will be used. Approximately 1×10⁶ cells in 30 μL of medium mixed with same volume (30 μL) of Matrigel was planted on the chorioallantoic membrane.

Microscopic analysis of CAM sections: After incubation at 37° C. with 55% relative humidity for 3 days, the CAM tissue directly beneath each filter disk will be resected from control and treated CAM samples. Tissues were washed 3 times with PBS, placed in 35-mm Petri dishes (Nalge Nunc, Rochester, N.Y.) and examined under an SV6 stereomicroscope (Karl Zeiss, Thornwood, N.Y.) at 50× magnification. Digital images of CAM sections exposed to filters will be collected, using a 3-CCD color video camera system (Toshiba America, New York, N.Y.), and analyzed with Image-Pro software (Media Cybernetics, Silver Spring, Md.). The numbers of vessel branch points contained in a circular region equal to the area of each filter disk will be counted. One image will be counted in each CAM preparation, and findings from 6-8 CAM preparations/group will be analyzed for each treatment condition.

Results presented as mean tumor weight (mg) per treatment group and tumor hemoglobin (mg/dL)±SD, n=8 eggs per group. The effect of these treatments was determined after 7 days of implantation. Results are presented as a mean tumor weight (g) per treatment group and tumor hemoglobin (mg/dl)±SD, n=8 per group.

### Example 29

### Anti-Cancer Efficacy in Athymic Nude Mice

Human Pancreatic cancer cells (MPanc) were implanted subcutaneously in nude mice at 1 million cells per site and after tumor growth to 150-200 mm3, animals were implanted with the various Nano composites near by the tumor implant. After 2 weeks, tumors were excised and weighed and relevant data is presented in Table 1.

TABLE 1
Anti-Cancer Efficacy of different polyphenol, thiol compounds alone or
co-encapsulated into Nano composites.

| | Mean |
|---|---|
| | Pancreatic Tumor |
| Treatment | Weight (mg) ± SD |
| Control | 291 ± 25 |
| Nano-Pomegranate extract (10 ug/implant) | 21.48 ± 7.2 |
| Nano-Punicalagin (10 ug/implant) | 51.68 ± 11.1 |
| Nano-Ellagic acid (10 ug/implant) | 48.03 ± 10.7 |
| Nano-Resveratrol (10 ug/implant) | 45.77 ± 9.8 |
| Nano-Sulforaphan (10 ug/implant) | 49.93 ± 8.8 |
| Nano-Ajoene (10 ug/implant) | 46.23 ± 11.1 |
| Nano-Resveratrol + Ajoene (10 ug/implant) | 11.03 ± 3.3 |
| Nano-Punicalagin + Sulforaphan (10 ug/implant) | 7.68 ± 2.5 |
| Nano-EGCG + allicin (10 ug/implant) | 4.89 ± 3.7 |
| Pomegranate extract (10 ug/implant) | 175.55 ± 18.9 |
| EGCG (10 ug/implant) | 145.11 ± 17.8 |
| Resveratrol (10 ug/implant) | 198.44 ± 18.9 |
| Ajoene (10 ug/implant) | 188.45 ± 15.6 |
| Sulforaphan (10 ug/implant) | 195.80 ± 21.2 |
| Punicalagin (10 ug/implant) | 146.44 ± 16.4 |
| Resveratrol + Ajoene (10 ug/implant) | 65.82 ± 10.8 |
| Punicalagin + Sulforaphan (10 ug/implant) | 57.23 ± 11.3 |
| EGCG + allicin (10 ug/implant) | 68.65 ± 12.8 |

The preceding data represents mean tumor weight±Standard Deviation (SD), n=6 per group, after 2 weeks of pancreatic cancer cell implant at 1 million per animals. Nano composites were implanted in a Matrigel near by the tumor implants

### Example 30

### Statistical Analysis

Statistical analysis will be performed using one-way ANOVA and comparing the mean±SD of branch points from each experimental group with its respective control group. Statistical differences approaching P<0.05 will be considered to be a statistically significant difference. In the CAM studies, the angiogenesis index for each treatment group will be compared with the corresponding vehicle treated control group.

### Example 31

### Anti-Infectious Effects of Ajwa Extract

1—Blood cultures were performed using automated blood culture system. A total of 5 mls of each patient's blood was inoculated into each pediatric bottle of blood culture system.
2—Culture bottles were loaded into BacT/Alert blood culture and remained there until designated positive for a maximum of 5 days incubation time.
3—All bottles designated positive were smeared for Gram stain and were processed and cultured on Blood agar, MacConkey agar and Chocalete agar (Saudi prepared media Laboratories, Riyadh, KSA). The cultured plates were incubated for 18-24 hours (MacConkey agar at 35-37° C. in ordinary incubator (Forma Scientific Incubator, Germany). Blood agar and chocolate agar plates were incubated at 35-37° C. in 5-10% CO2 incubator (Sanyo CO2 Incubator, Japan).
4—The colonies were identified by Gram staining for further identification (ID) and antibiotic sensitivity testing (AST) by Vitek® 2 system (bioMerieux, Inc., France).
5—The isolated pure colonies were selected and a purity plate was done to ensure that a pure culture was used for testing.
6—3 ml of 0.45% sterile saline was aseptically added into clear plastic test tube.
7—A sufficient number of morphologically similar colonies was transferred by a sterile loop to the saline tube and its density was checked by using Vitek 2 DensiCheck (bioMerieux, Inc., France), and equivalent to 0.5 to 0.63 McFarland.
8—The suspension tube was placed in the cassette followed by an empty tube.
9—The identification card was placed in the suspension tube and the AST card was placed in the empty tube.
10—When the sample cycle was finished, the cassettes and the tubes were discarded.
11—Minimal Inhibitory Concentration (MIC) was calculated and represented as (sensitive, intermediate or resistant).
12—IF the identified organism was acinetobater baumanii, the MIC of Amikacin and Meropenum were not done by Vitek 2 system, but done by E-test strips (bioMerieux SA, RCS LYON, Marcy-l' Etolle, France).
13—Culture bottles positive for yeast cells were cultured on to Sabouraud agar (SDA)(Saudi prepared media Laboratories, Riyadh, KSA) and the yeasts were identified with the use of VITEK MS at the same day of sufficient growth on SDA, then the identification (ID) is confirmed by using VITEK® 2 system for ID and antifungal susceptibility.

### Example 32

### Primary Yeast Identification by VITEK MS (bioMerieux, Inc., France VITEK MS)

Instrument employed is a matrix-assisted laser desorption ionization-time of flight mass spectrometer (MALDI-TOFMS) used as a rapid method for bacterial and fungal identification from microbial cultures.

A sufficient amount (about 3 mm) of yeast colonies isolated from young culture grown on SDA was taken and applied on the VITEK MS disposable slide (VITEK MS-DS). A total of 0.5 μl of VITEK MS-FA (formic acid) was applied on the sample and allowed to be evaporized within 1 to 3 minutes. Then, 1.0 μl of VITEK MS-CHCA matrix was added to the previous mixture and checked after 5 minutes for crystals to be visible. The targeted slide was placed on the adapter, and the adapter was loaded into the VITEK MS instrument for the analysis to be started. The sample was submitted to multiple laser shots inside the VITEK MS mass spectrometer. The sample spectra will be interpreted to provide organism identification results associated with a confidence level.

### Example 33

### Final Yeast Identification and Anti-Fungal Susceptibility Testing by VITEK-2 (bioMerieux, Inc., France)

The isolated pure colonies were selected from (SDA) and a purity plate was done to ensure that a pure culture was used for testing. 3 ml of 0.45% sterile saline was aseptically added into clear plastic test tube. A sufficient number of morphologically similar colonies was transferred by a sterile loop to the saline tube and its density was checked by using Vitek 2 DensiCheck (bioMerieux, Inc., France) which should be equivalent to (2) McFarland. Then, the suspension tube was placed in the cassette followed by an empty tube and the card for identification of yeast was placed in the suspension tube and the card for AST (AST-YS07) was placed in the empty tube. When the sample cycle was finished, the cassettes and the tubes were discarded. Minimal Inhibitory Concentration (MIC) was calculated and represented as (sensitive, intermediate or resistant).

Other specimens were processed and cultured on Blood agar, MacConkey agar and Chocalete agar (Saudi prepared media Laboratories, Riyadh, KSA) except urine specimens were processed and cultured on Blood agar and MacConkey agar.

The cultured plates were incubated for 18-24 hours (MacConkey agar at 35-37° C. in ordinary incubator; Forma Scientific Incubator, Germany). Blood agar and Chocalete agar plates were incubated at 35-37° C. in 5-10% CO2 incubator.

The colonies were identified by Gram staining to see Gram negative bacilli for further identification (ID) and antibiotic sensitivity testing (AST) by Vitek® 2 system (bioMerieux, Inc., France).

Table 2 shows distinct suppression of various microbial infection by Ajwa extract in various patient populations admitted to hospitals.

Fifty two pediatric cancer patients with the diagnosis of ALL (30/52), AML (10/52) and solid tumors (13/52). Group 1 included 26 patients 50% with a mean yearly admission 2.6 and reported negative cultures in 25 patients 96%, while the control group included 26 patients 500 with a mean yearly admissions 7.75 and reported positive cultures in 23 patients 88.5%, P value <0.001.

Additionally, Group 1 showed decreased cardiotoxicity compared to control.

In conclusion, dates decreased infections and morbidities among pediatric oncology patients.

TABLE 2
Suppression of infection in pediatric oncology patients on Ajwa extract

| | | Ajwa intake | | |
|---|---|---|---|---|
| | | Total | Yes (%) | No (%) |
| | | 55 | 32 | 23 |
| Infection | No | 23 (41.8%) | 23 (100.0%) | 0 (0.0%) |
| | Yes | 32 (58.2%) | 0 (0.0%) | 32 (100.0%) |

Table 2 shows that Ajwa extract intake resulted in 100% suppression of various types of infection versus placebo.

FIG. 1 depicts SEP Fraction eluted with 30% Methanol—HPLC chromatogram and UV spectrum of date seed extract fraction through XAD-7 resin, in accordance with embodiments of the present invention. NH2 column is eluted in gradient with 98-90% methanol containing 4 mM ammonium acetate and 0.1% formic acid, UV254 nm.

FIG. 2 depicts SEP Fraction eluted with 45% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid, UV254 nm.

FIG. 3 depicts SEP Fraction eluted with 60% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid, UV254 nm.

FIG. 4 depicts SEP Fraction eluted with 70% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid, UV254 nm.

FIG. 5 depicts SEP Fraction eluted with 100% Methanol—HPLC chromatogram and UV spectrum of date seed extract fraction through XAD-7 resin, in accordance with embodiments of the present invention. NH2 column eluted in gradient with 98-90% methanol containing 4 mM ammonium acetate and 0.1% formic acid, UV254 nm.

FIG. 6 depicts a mass spectrogram of 60% ethanol extract of date seed), in accordance with embodiments of the present invention. MS scanned from 100 through 1000 m/z in positive Q1 model. Comparing with the control matrix (95% methanol), these ion peak (in circle) only appear in 60% ethanol extract of date seed powder.

FIG. 7 depicts SIM (selected ion monitoring) plots of 60% ethanol extract of date seed powder, in accordance with embodiments of the present invention. LC conditions: NH2 column eluted in gradient with 98-90% methanol containing 4 mM ammonium acetate and 0.1% formic acid, three peaks corresponded with the unique masses found by MS scan.

FIG. 8 depicts chemical structure and schematic illustration of OEGCG synthesized from the inter-molecular poly condensation reaction of EGCG, in accordance with embodiments of the present invention. FIG. 8 is a schematic of the self-assembly process used to form the Lycopene/OEGCG/Chitosan or Methylated Chitosan NPs, which are formed via two sequential self-assemblies processes in an aqueous solution, complexation of OEGCG with lycopene to form the core, followed by coating with chitosan to form the shell.

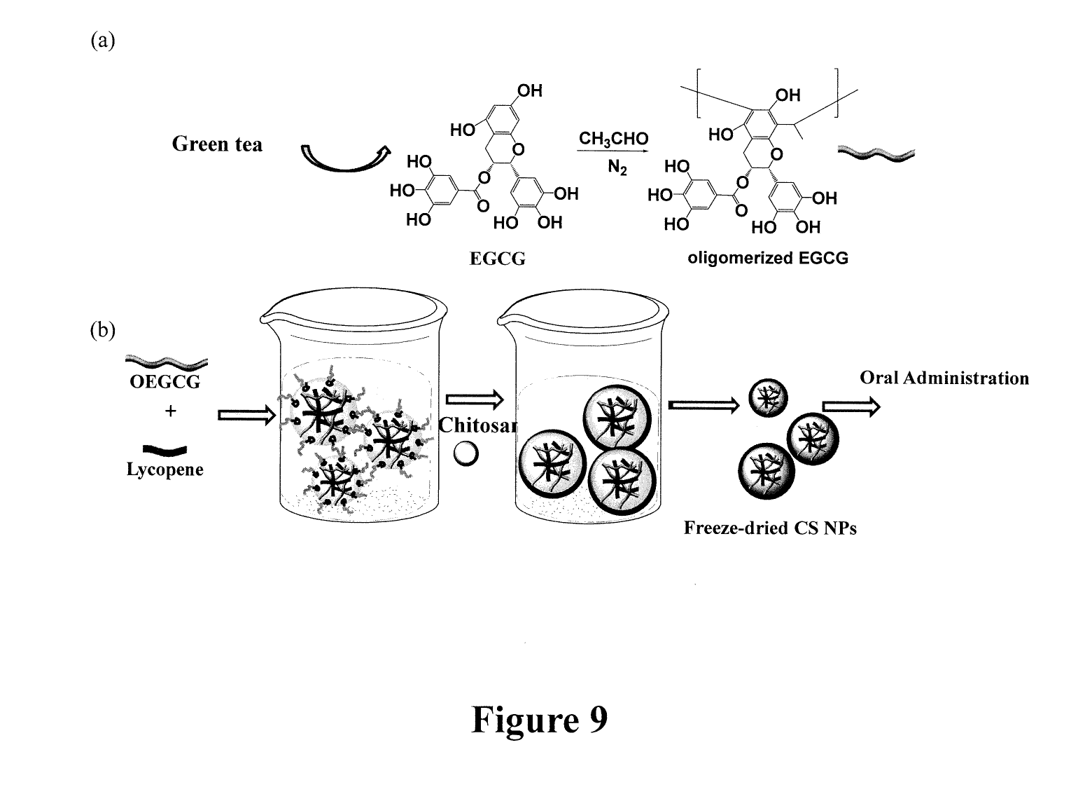

FIG. 9 depicts chemical structure and schematic illustration of OEGCG synthesized from the intermolecular poly condensation reaction of EGCG, in accordance with embodiments of the present invention. FIG. 9 is a schematic of the self-assembly process used to form the Lycopene/OEGCG/Chitosan or methylated Chitosan NPs, which are formed via two sequential self-assemblies processes in an aqueous solution, complexation of OEGCG with lycopene to form the core, followed by coating with chitosan to form the shell. Freeze drying of this nanoparticle was also evaluated as a means to improve shelf life. Then, formulations were administered by oral gavage into mice.

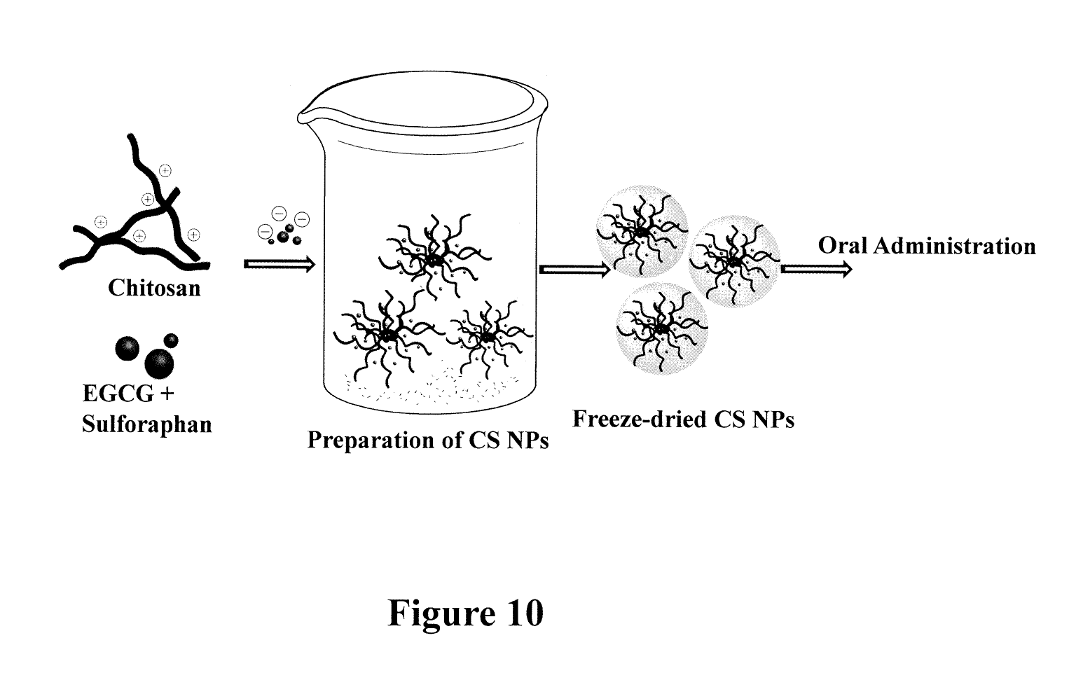

FIG. 10 depicts the preparation of freeze-dried CS NPs, in accordance with embodiments of the present invention. The prepared Chitosan-coated NPs can prevent the release of EGCG/Sulforaphan from CS NPs in the stomach and enhance their absorption on the surface of the small intestine, thus further increasing their bioavailability. Freeze drying of this nanoparticle was also evaluated as a means to improve shelf life. Then, formulations were administered by oral gavage into mice. CS NPs, chitosan nanoparticles.

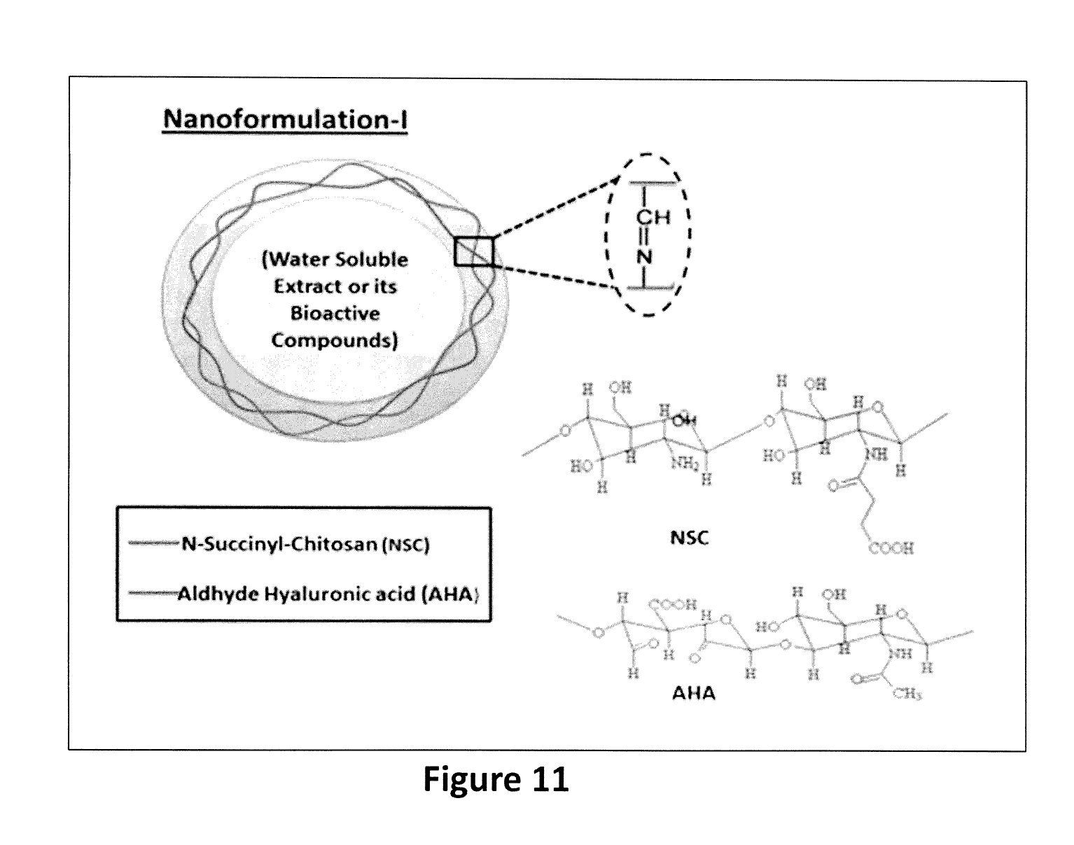

FIG. 11 depicts complex formation of Chitosan derivative and Hyaluronic acid derivative for encapsulation of water soluble extract or bioactive compounds thereof, in accordance with embodiments of the present invention.

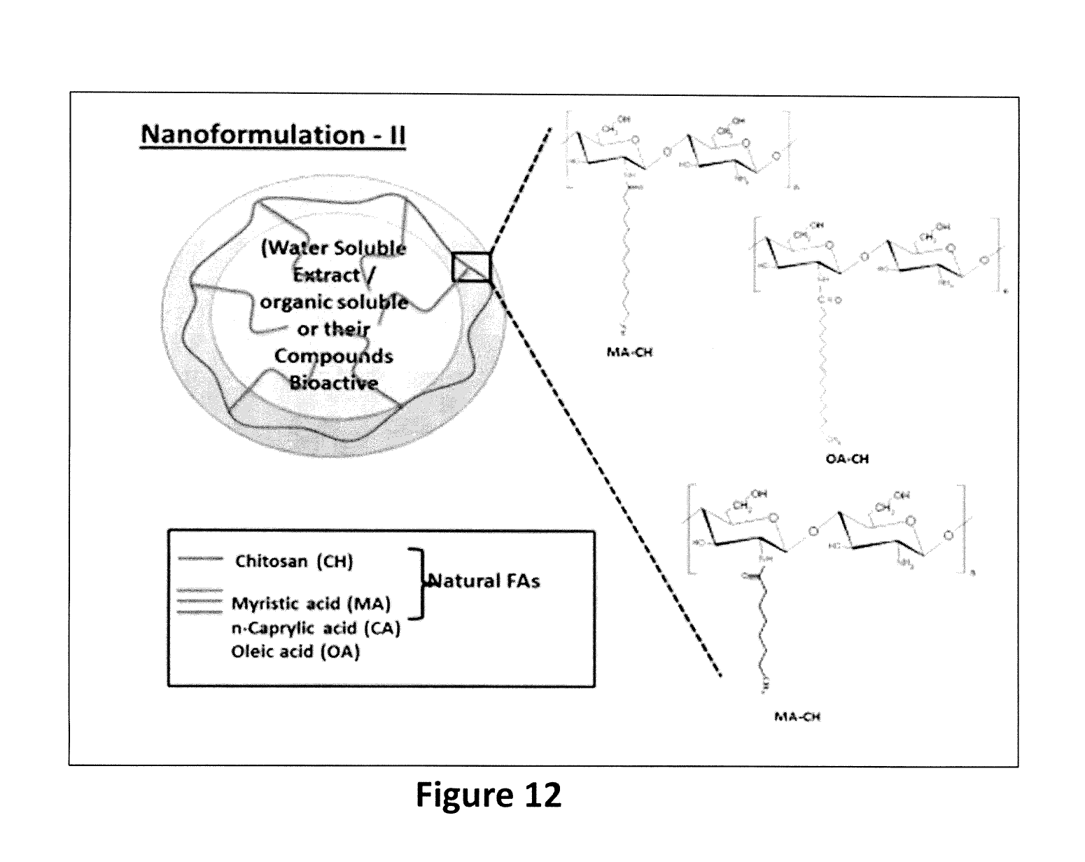

FIG. 12 depicts nanoformulation of complex formation of Chitosan and Oleic acid, Myristic acid, and Caprylic acid for encapsulation of water soluble or insoluble extract and bioactive compounds thereof, in accordance with embodiments of the present invention.

FIG. 13A depicts procedures for preparation of dates flesh extract of bioactive compounds, in accordance with embodiments of the present invention.

FIG. 13B depicts procedures for preparation of dates seed extract, in accordance with embodiments of the present invention.

FIG. 13C depicts procedures for extraction and HPLC analysis for date's seed powder, in accordance with embodiments of the present invention.

FIG. 14A depicts MS-MS spectra of subsequent fragment ions of punicalagin: punicalin and gallagic acid, in accordance with embodiments of the present invention.

FIG. 14B depicts spectra of the molecular ion of ellagic acid and its subsequent ions, in accordance with embodiments of the present invention.

FIG. 14C depicts HPLC chromatogram of punicalagin and gallagic acid separated on C18 column (Waters, Sunfire, 3.0×150 mm, 5 μm), with mobile phase acetonitrile and 0.1 formic acid and UV 366 nm, in accordance with embodiments of the present invention. AU is area under the curve.

FIG. 15A depicts structure of naturally driven thiols such as allicin, ajoene (E and Z), sulforaphan, and conjugated sulforaphan, in accordance with embodiments of the present invention.

FIG. 15B depicts structure of naturally driven polyphenols including flavonoids, isoflavone, lignans, stilbenes, and other polyphenols, in accordance with embodiments of the present invention.

FIG. 15C depicts structure of naturally driven punicalagin, ellagic acid, gallagic acid, and other polyphenols, in accordance with embodiments of the present invention.

FIG. 16 is a scheme for sample preparation for LC/MS/MS, in accordance with embodiments of the present invention. Mouse plasma was mixed with formic acid (FA) and methanol (MeOH), vortexed, centrifuged and supernatant was dried under nitrogen. The dried supernatant was re-suspended with 75% methanol and 0.2% FA, and then the sample was injected into the LC/MS/MS.

FIG. 17 depicts a LC/MS/MS chromatogram illustrating distinct differences in the detectable levels of Ellagic acid in plasma from mice treated with Nano-Pomegranate extract versus un-detectable levels in plasma from animals treated with plain Pomegranate extracts, in accordance with embodiments of the present invention.

FIG. 18 depicts plasma Levels of ellagic acid in mice treated with plain pomegranate extract (n=4) versus Nano pomegranate extract (n=4), in accordance with embodiments of the present invention. The data showed a distinct differences in the detectable levels of Ellagic acid in plasma from animals treated with Nano-Pomegranate extract (Area Under the Curve, AUC ranged from 2,000-20,000) versus very low to un-detectable levels (AUC ranged from 200 to 1,000) in plasma from animals treated with plain Pomegranate extracts.

FIG. 19 depicts procedures for resveratrol extraction in plasma using Solid Phase Extraction, in accordance with embodiments of the present invention. Blood samples were taken into heparinized capillaries at 5, 15, 30, 60, 180, and 360 minutes post-dosing. Extraction of resveratrol spiked in plasma versus plasma from treated animals was carried out as shown in the scheme in FIG. 19.

FIG. 20A depicts a resveratrol plasma concentration time curve in mice for subcutaneously injected plain resveratrol versus subcutaneously injected nano-resveratrol analyzed by LC/MS/MS method, in accordance with embodiments of the present invention. The data showed greater levels in plasma from animals treated with nano-resveratrol versus those treated with plain resveratrol subcutaneously.

FIG. 20B depicts resveratrol concentration in tissues such as liver homogenate (n=3) of mice from oral delivery and subcutaneously injection of nano-resveratrol into the mice liver of mice treated with nano-resveratrol, in accordance with embodiments of the present invention. FIG. 20B shows that the resveratrol concentration from the oral delivery of nano-resveratrol was much greater the resveratrol concentration in those animals treated with plain resveratrol after either subcutaneous or oral administration.

FIG. 21 depicts effect of Resveratrol, Z-Ajoene, Resveratrol/Z-Ajoene Nano, in accordance with embodiments of the present invention. Data showed synergistic anti-angiogenesis efficacy of co-encapsulated Resveratrol/Z-Ajoene in the CAM model of FGF-mediated angiogenesis.

The present invention provides a nano-composition comprising nanoparticles. Each nanoparticle comprises a nano-shell and one or more compounds co-encapsulated within the nano-shell. The nano-shell of each nanoparticle comprises one or more chitosan polymers and one or more polymers subject to each chitosan polymer being covalently bonded to the one or more polymers. Each chitosan polymer in the nano-shell of each nanoparticle is independently selected from the group consisting of chitosan, tri-methylated chitosan, and a combination thereof. The one or more polymers in the nano-shell of each nanoparticle comprise poly(lactide-co-glycolide) (PLGA), one or more fatty acids, or combinations thereof subject to each fatty acid being independently selected from the group consisting of oligomer epigallocatechin-3-gallate (OEGCG), hyaluronic acid, oleic acids, myristic acid, caprylic acid, and combinations thereof. Each compound co-encapsulated within the nano-shell is independently selected from the group consisting of ajwa extracts, pomegranate extracts, garlic extracts, one or more polyphenols, one or more thiols, and combinations thereof.

In one embodiment, the nanoparticles of the nano-composition are coated with one or more targeting moieties covalently bonded to respective nano-shells.

In one embodiment, the one or more targeting moieties are independently selected from the group consisting of glycyrrhizin, glycyrrhizic acid, glycyrrhizinic acid, and combinations thereof.

In one embodiment, each compound co-encapsulated within the nano-shell is covalently bonded to the one or more chitosan polymers and/or the one or more polymers for sustained release of the one or more compounds co-encapsulated within the nano-shell.

In one embodiment, one or more chitosan polymers comprise 50 to 90 percent by weight of the nano-shell of each nanoparticle.

In one embodiment, one or more chitosan polymers comprise 10 to less than 50 percent by weight of the nano-shell of each nanoparticle.

In one embodiment, the one or more polymers in the nano-shell of each nanoparticle comprise the PLGA.

In one embodiment, the one or more polymers in the nano-shell of each nanoparticle comprise the one or more fatty acids.

In one embodiment, the one or more compounds co-encapsulated within the nano-shell of each nanoparticle comprise the one or more polyphenols, and wherein the one or more polyphenols are selected from the group consisting of epigallocatechin-3-gallate (EGCG), resveratrol, ellagic acid, and combinations thereof.

In one embodiment, the one or more compounds co-encapsulated within the nano-shell of each nanoparticle comprise the one or more thiols, and wherein the one or more thiols are selected from the group consisting of ajoene, allicin, sulforaphan, and combinations thereof.

In one embodiment, the one or more compounds co-encapsulated within the nano-shell of each nanoparticle comprise the ajwa extracts, and wherein the ajwa extracts are water soluble, wherein the one or more chitosan polymers comprise chitosan, and wherein and the one or more polymers comprise hyaluronic acid.

In one embodiment, the one or more compounds co-encapsulated within the nano-shell of each nanoparticle comprise the ajwa extracts, wherein the ajwa extracts are water insoluble, wherein the one or more chitosan polymers comprise chitosan, and wherein and the one or more polymers are selected from the group consisting of oleic acids, myristic acid, caprylic acid, and combinations thereof.

In one embodiment, each chitosan polymer has a molecular weight in a range of 5,000 to 1200,000 Daltons.

In one embodiment, the nanoparticles have a linear size in a range of 150 to 500 nm.

In one embodiment, the nanoparticles have a positive zeta potential in a range of +10 to +30 mv or a negative zeta potential in a range of −10 to −30 mv.

The present invention provides a method of forming the nano-composition, the method comprising: forming the nano-shell of each nanoparticle; and co-encapsulating the one or more compounds within the nano-shell of each nanoparticle.

In one embodiment, the method further comprises: lyophilizing the nanoparticles; and prior to lyophilizing the nanoparticles, adding mannitol or sucrose as a cryoprotectant to the nanoparticles.

The present invention provides a method of using the nano-composition, the method comprising: administering the nano-composition to a mammal (e.g., human being).

In one embodiment, the mammal (e.g., human being) has a chemo-preventive disorders.

In one embodiment in which the mammal (e.g., human being) has a chemo-preventive disorder, the nano-composition is a chemo-preventive promoting anti-angiogenesis functionality, anti-cancer functionality, efficacy and safety for chemotherapy and/or radiotherapy, or combinations thereof.

In one embodiment in which the mammal (e.g., human being) has a chemo-preventive disorder, the nano-composition is a chemo-preventive functioning to prevent hepatic disorders, pulmonary disorders, cardiac disorders, kidney fibrosis disorders, or combinations thereof.

In one embodiment, administering the nano-composition comprises administering the nano-composition orally, topically, or by injection.

While particular embodiments of the present invention have been described herein for purposes of illustration, many modifications and changes will become apparent to those skilled in the art. Accordingly, the appended claims are intended to encompass all such modifications and changes as fall within the true spirit and scope of this invention.

What is claimed is:

1. A nano-composition comprising: nanoparticles,
wherein each nanoparticle comprises a nano-shell and one or more compounds co-encapsulated within the nano-shell,
wherein the nano-shell of each nanoparticle comprises one or more chitosan polymers and one or more polymers subject to each chitosan polymer being covalently bonded to the one or more polymers,
wherein each chitosan polymer in the nano-shell of each nanoparticle is independently selected from the group consisting of chitosan, tri-methylated chitosan, and a combination thereof,
wherein the one or more polymers in the nano-shell of each nanoparticle comprise poly(lactide-co-glycolide) (PLGA), one or more fatty acids, or combinations thereof subject to each fatty acid being independently selected from the group consisting of oligomer epigallocatechin-3-gallate (OEGCG), hyaluronic acid, oleic acids, myristic acid, caprylic acid, and combinations thereof, and
wherein each compound co-encapsulated within the nano-shell is independently selected from the group consisting of ajwa extracts, pomegranate extracts, garlic extracts, one or more polyphenols, one or more thiols, and combinations thereof.

2. The nano-composition of claim 1, wherein the nanoparticles of the nano-composition are coated with one or more targeting moieties covalently bonded to respective nano-shells.

3. The nano-composition of claim 2, wherein the one or more targeting moieties are independently selected from the group consisting of glycyrrhizin, glycyrrhizic acid, glycyrrhizinic acid, and combinations thereof.

4. The nano-composition of claim 1, wherein each compound co-encapsulated within the nano-shell is covalently bonded to the one or more chitosan polymers and/or the one or more polymers for sustained release of the one or more compounds co-encapsulated within the nano-shell.

5. The nano-composition of claim 1, wherein one or more chitosan polymers comprise 50 to 90 percent by weight of the nano-shell of each nanoparticle.

6. The nano-composition of claim 1, wherein one or more chitosan polymers comprise 10 to less than 50 percent by weight of the nano-shell of each nanoparticle.

7. The nano-composition of claim 1, wherein the one or more polymers in the nano-shell of each nanoparticle comprise the PLGA.

8. The nano-composition of claim 1, wherein the one or more polymers in the nano-shell of each nanoparticle comprise the one or more fatty acids.

9. The nano-composition of claim 1, wherein the one or more compounds co-encapsulated within the nano-shell of each nanoparticle comprise the one or more polyphenols, and wherein the one or more polyphenols are selected from the group consisting of epigallocatechin-3-gallate (EGCG), resveratrol, ellagic acid, and combinations thereof.

10. The nano-composition of claim 1, wherein the one or more compounds co-encapsulated within the nano-shell of each nanoparticle comprise the one or more thiols, and wherein the one or more thiols are selected from the group consisting of ajoene, allicin, sulforaphan, and combinations thereof.

11. The nano-composition of claim 1, wherein the one or more compounds co-encapsulated within the nano-shell of each nanoparticle comprise the ajwa extracts, and wherein the ajwa extracts are water soluble, wherein the one or more chitosan polymers comprise chitosan, and wherein and the one or more polymers comprise hyaluronic acid.

12. The nano-composition of claim 1, wherein the one or more compounds co-encapsulated within the nano-shell of each nanoparticle comprise the ajwa extracts, wherein the ajwa extracts are water insoluble, wherein the one or more chitosan polymers comprise chitosan, and wherein and the one or more polymers are selected from the group consisting of oleic acids, myristic acid, caprylic acid, and combinations thereof.

13. The nano-composition of claim 1, wherein each chitosan polymer has a molecular weight in a range of 5,000 to 1200,000 Daltons.

14. The nano-composition of claim 1, wherein the nanoparticles have a linear size in a range of 150 to 500 nm.

15. The nano-composition of claim 1, wherein the nanoparticles have a positive zeta potential in a range of +10 to +30 mv or a negative zeta potential in a range of −10 to −30 mv.

16. A method of forming the nano-composition of claim 1, said method comprising:
forming the nano-shell of each nanoparticle; and
co-encapsulating the one or more compounds within the nano-shell of each nanoparticle.

17. The method of claim 16, said method further comprising:
lyophilizing the nanoparticles; and
prior to said lyophilizing the nanoparticles, adding mannitol or sucrose as a cryoprotectant to the nanoparticles.

18. A method of using the composition of claim 1, said method comprising:
administering the nano-composition to a human being.

19. The method of claim 18, wherein the human being has a chemo-preventive disorder.

20. The method of claim 19, wherein the nano-composition is a chemo-preventive promoting anti-angiogenesis functionality, anti-cancer functionality, efficacy and safety for chemotherapy and/or radiotherapy, or combinations thereof.

21. The method of claim 19, wherein the nano-composition is a chemo-preventive functioning to prevent hepatic disorders, pulmonary disorders, cardiac disorders, kidney fibrosis disorders, or combinations thereof.

22. The method of claim 18, wherein said administering comprises administering the nano-composition orally, topically, or by injection.
